(19) United States
(12) Patent Application Publication
(10) Pub. No.: US 2009/0118248 A1
(43) Pub. Date: May 7, 2009

(54) 3-Alpha-hydroxy 21-n-heteroaryl-pregnane derivatives for modulation of brain excitability and a process for the production thereof

(75) Inventors: Ping W. Chang, Waterford, CT (US); Ning Zhong, Flushing, NY (US); Xinping Fang, Closter, NJ (US); Shao-Kun Pang, San Diego, CA (US); Chi-Nung Hsiao, Libertyville, IL (US); Tsung-Cheng Hu, Changhua County (TW)

Correspondence Address: STERNE, KESSLER, GOLDSTEIN & FOX P.L.L.C., 1100 NEW YORK AVENUE, N.W., WASHINGTON, DC, 20005, US

(73) Assignee: Euro-Celtique S.A., Luxembourg (LU)

(21) Appl. No.: 11/587,138

(22) PCT Filed: Apr. 22, 2005
(86) PCT No.: PCT/US2005/014028

§ 371 Date: Jan. 7, 2009

Related U.S. Application Data
(60) Provisional application No. 60/564,652, filed on Apr. 23, 2004.

(51) Int. Cl. A61K 31/58; C07J 7/00; A61P 25/00; C07J 43/00

(52) U.S. Cl. 514/176; 552/609; 540/111

(57) ABSTRACT

The invention relates to a novel multi step process of making compounds of Formula I:

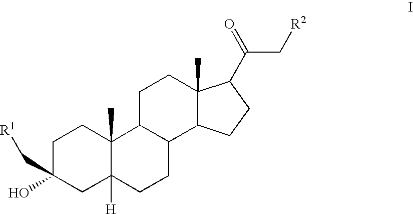

wherein R¹ is an alkoxy group and R² is an optionally substituted, N-attached heteroaryl. The hydrogen at the 5-position can be α or β isomer, preferably α. Preferably the compound of Formula I is 17β isomer. The invention also relates to novel 3α-hydroxy-3β-substituted-17-substituted steroid compounds having GABA_{A} receptor modulating activity, pharmaceutical compositions comprising these compounds, and the use of these compounds in a method of modulating brain excitability.

### BACKGROUND OF THE INVENTION

1. Field of the Invention

The present invention relates to the field of medicinal chemistry. Specifically, the present invention relates to a process for preparing 3α-hydroxy-3β-alkoxymethyl-21-substituted-5α (and 5β)-pregnan-20-ones. Further, the present invention relates to novel steroid derivatives with properties desirable for use as sedative/hypnotics, as anxiolytics, and for inducing anesthesia.

2. Related Art

International Published Application WO 93/18053 describes a method of making 3α-hydroxy-3β-substituted-pregnanes by converting pregnan-3,20-dione compounds into a 3(R)-pregnan-3-spiro-2′-oxirane-20-one intermediate, and then converting the intermediate into 3α-hydroxy-3β-substituted-pregnanes by selectively opening the oxirane ring using a suitable nucleophile. Suitable nucleophiles are described to include alkoxides, thioalkoxides, azides, cyanide, isocyanide, amines and halide anions such as iodide. Specifically, WO 93/18053 describes the use of NaI in anhydrous 1,2-dimethoxyethane or in glacial acetic acid and 50:50 tetrahydrofuran/methanol in the oxirane ring opening reaction.

International Published Application WO 95/21617 describes compounds of the following Formula:

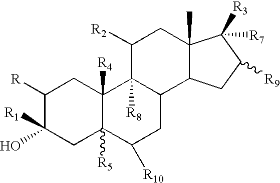

wherein R₁ can be, e.g., alkoxyalkynyl and R₃ can be an optionally substituted heteroarylacetyl group. These compounds can be prepared by allowing a suitable 3α-hydroxy-5β-pregnan-20-one derivative to react with bromine in methanol to obtain the 21-bromo-derivative and allowing this compound to react with a suitable heteroaryl in an inert atmosphere. The compounds are described to modulate GABA receptor activity and, therefore, to be useful as anticonvulsants, sedative/hypnotics, anxiolytics, and anesthetics.

International Published Application WO 00/66614 describes compounds of the following Formula:

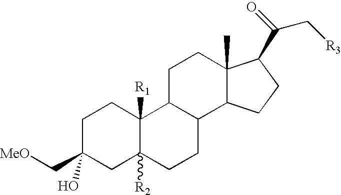

wherein R₁ is hydrogen or methyl, R₂ is 5α- or 5β-hydrogen, and R₃ can be an optionally substituted, N-attached heteroaryl group, such as an imidazolyl group. These compounds can be prepared by brominating 3α-hydroxy-3β-(methoxymethyl)-5α-pregnan-20-one with bromine in the presence of a catalytic amount of a 48% HBr solution to produce a mixture containing 21-bromo-3α-hydroxy-3β-(methoxymethyl)-5α-pregnan-20-one, and reacting the mixture with a heteroaryl, such as imidazole, at reflux temperature having CH₃CN as a solvent. These compounds are also described as being useful as sedatives/hypnotics and anesthetics.

Hogenkamp et al. describe the ring opening of (3R)-spiro[oxirane-2′,5α-pregnan]-20-one in a methanol solution by adding sodium (Na) metal to the solution and refluxing for 16 hours to produce an 84:16 mixture of 3α-hydroxy-3β-(methoxymethyl)-5α-pregnan-20-one and its 17α epimer (J. Med. Chem. 40:61-72 (1997)).

The conventional methods for preparing 3α-hydroxy-3β-alkoxymethyl-21-substituted-5α-pregnan-20-ones suffer from limited product yields resulting from incomplete reactions and inefficient purification procedures required to reduce the level of impurities. 3α-Hydroxy-3β-alkoxymethyl-21-substituted-5α-pregnan-20-ones are useful as pharmaceutical agents that treat a number of conditions and, therefore, product purity is a concern. Attempts are being made to improve yields and to efficiently produce more pure products. Manufacturing difficulties are particularly acute when very expensive chiral starting materials are used. It can be readily seen that even minor improvements in process efficiency will result in economic benefits. This is particularly true upon scaleup manufacture of chiral products. A need therefore exists for processes of synthesis having improved yields, shorter reaction times and purer products, and that can be practiced on an industrially useful scale.

### SUMMARY OF THE INVENTION

The present invention relates to an improved multistep process for preparing 3α-hydroxy-3β-alkoxymethyl-21-substituted-pregnan-20-ones having the Formula I:

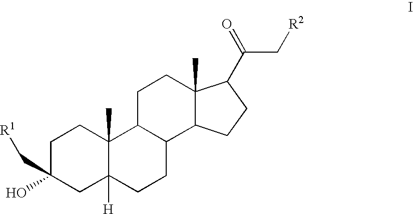

wherein:

R¹ is an alkoxy group; and

R¹ is an optionally substituted, N-attached heteroaryl. The hydrogen at the 5-position can be α or β isomer, and preferably α. Preferably, the compound of Formula I is 17β isomer.

In one aspect, the invention provides a process, comprising the step of reacting a compound of Formula II:

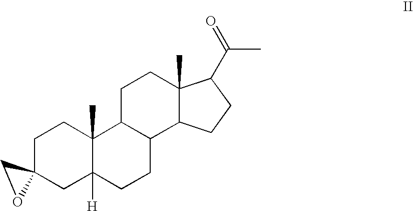

with a reagent comprising one or more alkali metal alkoxide, alkaline-earth metal alkoxide or alkali metal hydroxide, and optionally a Lewis acid, in an appropriate solvent to open the oxirane ring without affecting the 20-position keto group, to provide a reaction mixture comprising a compound of Formula III:

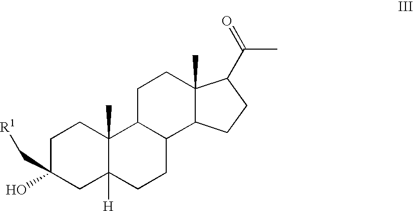

wherein R¹ is as defined above, and optionally isolating the desired 17β or 17α isomer. Preferably, the compound of Formula II is allowed to react with NaOMe or NaOH, more preferably NaOH, in methanol to provide a compound of Formula III wherein R¹ is a methoxy group. Preferably, the compound of Formula III is 5α isomer. Preferably, the 17β isomer of the compound of Formula III is isolated and purified by re-crystallization. Preferably, the 17α isomer is epimerized to obtain the 17β isomer, and the 17β isomer is re-crystallized.

In another aspect, the present invention provides a process comprising the step of reacting a compound of Formula III with a bihalogen in the presence of a haloacid to form a product mixture comprising a halogenated derivative having the Formula IV:

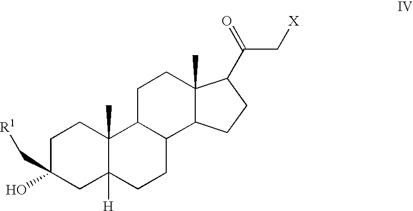

wherein

R¹ is as defined above and X is a halogen, and optionally isolating and purifying the compound of Formula IV. Preferably, the compound of Formula IV is 5α and 17β isomer, and purified up to about 80% purity (area percent as determined by HPLC).

In a further aspect, the present invention provides a process comprising the step of reacting a product mixture comprising a compound of Formula IV or reacting an isolated compound of Formula IV with a nitrogen-containing optionally substituted heteroaryl compound or an alkali metal salt thereof to form a compound of Formula I.

Preferably, the compound of Formula I produced by the process of the present invention is 3α-hydroxy-21-(1′-imidazolyl)-3β-methoxymethyl-5α-pregnan-20-one of the following formula:

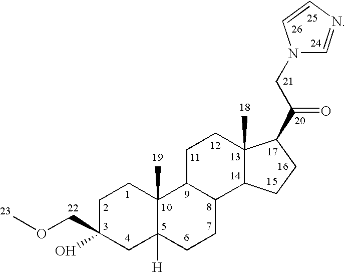

In a further aspect, the present invention provides a multistep process for preparing 3α-hydroxy-21-(1′-imidazolyl)-3-methoxymethyl-5α-pregnan-20-one. Accordingly, in one aspect, the present invention provides a process comprising reacting 5(3R)-spiro[oxirane-2′,5α-pregnan]-20-one of formula:

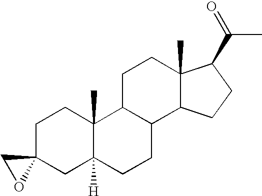

with NaOMe or NaOH in an appropriate solvent to obtain a mixture comprising 3α-hydroxy-3β-methoxymethyl-5α-pregnan-20-one of the formula

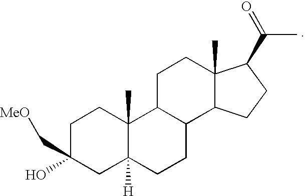

Preferably, the solvent is methanol. Preferably, the reaction temperature is about 35° C. to about 45° C. when 5(3R)-spiro[oxirane-2′,5α-pregnan]-20-one is reacted with NaOH in methanol. Preferably, the reaction temperature is reflux temperature when 5(3R)-spiro[oxirane-2′,5α-pregnan]-20-one is reacted with NaOMe in methanol. Preferably, the 17β isomer is purified from the mixture before using it in the next step.

In another aspect, the present invention provides a process comprising reacting 3α-hydroxy-3β-methoxymethyl-5α-pregnan-20-one of the formula

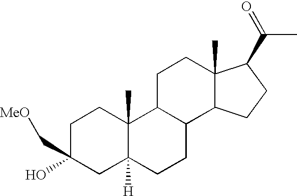

with Br₂ in the presence of HBr to obtain a mixture comprising a compound 21-bromo-3α-hydroxy-3β-methoxymethyl-5α-pregnan-20-one of formula

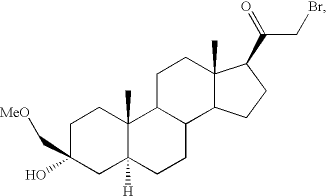

isolating the compound from the mixture, and optionally purifying the compound.

In a further aspect, the present invention provides a process for preparing 3α-hydroxy-21-(1′-imidazolyl)-3β-methoxymethyl-5α-pregnan-20-one of formula

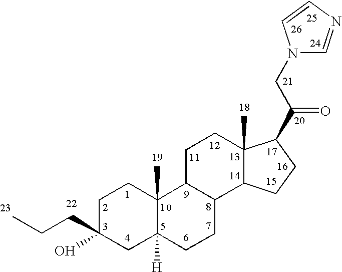

comprising reacting 21-bromo-3α-hydroxy-3β-methoxymethyl-5α-pregnan-20-one of formula

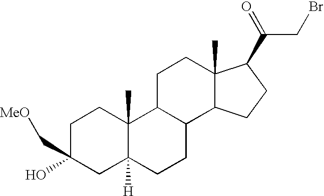

with a lithium salt or sodium salt of imidazole to obtain a mixture comprising the product 3α-hydroxy-21-(1′-imidazolyl)-3β-methoxymethyl-5α-pregnan-20-one. Preferably, Li-imidazole, i.e., lithium salt of imidazole, is used in this process.

The present invention also provides novel 3α-hydroxy-3β-substituted-17-substituted steroid compounds of Formula VIII, X, and XI, or pharmaceutically acceptable salts, prodrugs or solvates thereof having GABA_{A} receptor modulating activity.

The present invention further provides 3α-hydroxy-3β-substituted-17-substituted steroid compounds of Formula XII and XIII, or pharmaceutically acceptable salts or solvates thereof having GABA_{A} receptor modulating activity.

Further, the present invention provides a pharmaceutical composition comprising an effective amount of a compound of Formula VIII, X, XI, XII or XIII or a pharmaceutically acceptable salt or solvate thereof and one or more pharmaceutically acceptable carrier or diluent.

Furthermore, the present invention provides a method for modulating brain excitability by administering an effective amount of a compound of Formula VIII, X, XI, XII or XIII or a pharmaceutically acceptable salt or solvate thereof to a mammal in need of such treatment.

Additional embodiments and advantages of the invention will be set forth in part in the description as follows, and in part will be obvious from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION OF THE INVENTION

One aspect of the present invention is a new multistep process for preparing 3α-hydroxy-3β-alkoxymethyl-21-substituted-pregnan-20-ones having the Formula I:

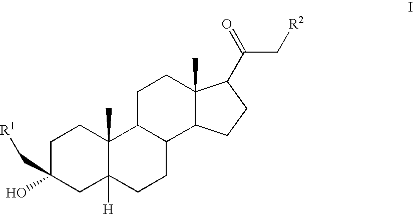

wherein:

R¹ is an alkoxy group; and

R² is an optionally substituted, N-attached heteroaryl. The hydrogen at the 5-position can be α or β isomer, and preferably α. Preferably, the compound of Formula I is 17β isomer.

It has been discovered that the process for preparing compounds of Formula I is improved, giving the steroid compound in a higher yield and/or with increased purity, when the process comprises the following step (a) reacting a compound of Formula II:

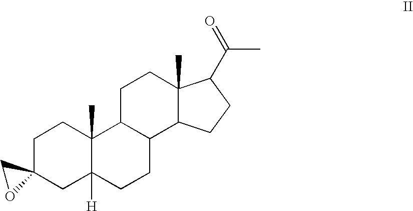

with a reagent comprising one or more alkali metal alkoxide, alkaline-earth metal alkoxide, or alkali metal hydroxide, and optionally a Lewis acid, in an appropriate solvent to open the oxirane ring without affecting the 20-position keto group, to provide a reaction mixture comprising a compound of Formula III:

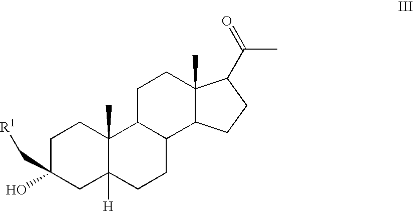

wherein R¹ is as defined above. Preferably, compounds of Formulae II and III are 5α isomers.

Suitable alkali metal alkoxides or alkaline-earth metal alkoxides include alkali metal or alkaline-earth metal C₁₋₆ alkoxides, preferably alkali metal or alkaline-earth metal C₁₋₄ alkoxides, such as methoxides, ethoxides, propoxides, iso-propoxides, butoxides, and tert-butoxides. Any alkali metal or alkaline-earth metal can be used to make the alkali metal or alkaline-earth metal alkoxides to be used in this step of the invention. Suitable alkali metals or alkaline-earth metals include lithium, sodium, magnesium, and calcium. Sodium is a particularly preferred alkali metal. Particularly preferred alkali metal alkoxides include sodium methoxide, sodium ethoxide, sodium propoxide, and sodium tert-butoxide, with sodium methoxide being particularly preferred.

Suitable alkali metal hydroxides include sodium hydroxide, lithium hydroxide, and potassium hydroxide, with sodium hydroxide being preferred.

Suitable solvents that can be used in the oxirane ring opening reaction include methanol or a mixture of methanol and an aprotic, polar solvent, such as tetrahydrofuran (THF), dimethoxyethane (DME), dimethyl formamide (DMF), dimethyl acetamide (DMAC), dimethyl sulfoxide (DMSO), diglyme, dioxane, or 1-methyl-2-pyrrolidinone. If a mixture of solvents is used, preferably 4 parts of MeOH is used with 1 part of an aprotic, polar solvent.

The reaction of step (a) can be conducted at reflux temperature or lower. Preferably, the reaction is conducted at about 25° C. to about 65° C., more preferably at about 35° C. to about 45° C. The reaction time can vary from 3 to 15 hours depending on the reaction temperature and the nature of the reagent used. Typically, at the reflux temperature, the reaction time is about 3-8 hours, and at about 35-45° C. the reaction time is from about 8 to 15 hours, depending on the nature of the reagent used.

Preferably, the reaction of step (a) is conducted under an inert atmosphere, such as under nitrogen or argon gas, and preferably under nitrogen gas.

The reaction of step (a) produces a mixture of 17β and 17α isomers of compounds of Formula III. Useful ratios of 17β:17α, include about 75:25, about 79:20, about 80:20, about 85:15, about 86:10, about 88:8, about 88:12, about 90:10, and about 94:5.

In a preferred embodiment, the reaction of step (a) is conducted by reacting a compound of Formula II with NaOH in methanol. Advantageously, the temperature of this reaction mixture is maintained at about 35-45° C. This process allows a mild ring opening with reduced amount of by-products and giving an isomer mixture of 17β and 17α having an improved ratio with regard to the 17β isomer.

Advantageously, the reaction mixture comprising the compound of Formula III is further treated to isolate the 17β isomer of Formula III and, optionally, to purify the 17β isomer by, e.g., recrystallization before further reaction steps. The 17β isomer of Formula III can be re-crystallized from a mixture of ethyl acetate and heptanes (v:v 1:1). The 17α isomer of Formula III can be further reacted to obtain the 17α isomer compounds of Formula I or epimerized and recycled, i.e., the produced 17β isomer of Formula III is recrystallized as described above. The epimerization can be conducted by, e.g., refluxing in MeOH in the presence of potassium carbonate (K₂CO₃). The epimerized mixture can be re-crystallized to obtain the 17β isomer of compounds of Formula III.

In another aspect of the present invention, the process of preparing 3α-hydroxy-3β-alkoxymethyl-21-substituted-pregnan-20-ones of Formula I comprises the step of

(b) reacting a compound of Formula III with a bihalogen in the presence of a haloacid to form a product mixture comprising a halogenated compound having the Formula IV:

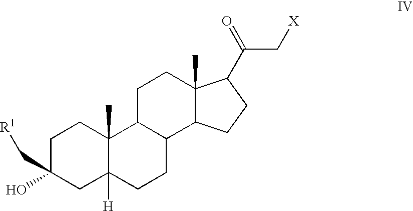

wherein R¹ is as defined above and X is halogen. Preferably, compounds of Formula IV are 5α isomers. In order to produce the 17β isomers of compounds of Formula IV, the starting compound of Formula III used in this step (b) is primarily the 17β isomer in order to obtain industrially useful yields.

Reaction conditions known in the art can be used in the halogenation reaction. Suitably, the reaction is conducted at room temperature and the reaction mixture is shielded from light. Suitable haloacids for use in step (b) include HCl, HF, HBr and HI. A particularly preferred haloacid is HBr. The haloacid initiates the halogenation reaction, and the absence of a catalytic amount of a haloacid increases the reaction time and generates by-products. Suitable bihalogens to be used in the reaction step (b) include Br₂ and Cl₂ in a suitable solvent. Advantageously, bromine in a methanol solution is used in the halogenation reaction. Bromine in methanol is made by adding Br₂ slowly to methanol at room temperature.

The product mixture of step (b) can be used as such in the next step (c) or it can be isolated and purified. Advantageously, in order to minimize the amount of by-products, the compound of Formula IV is isolated from the product mixture of step (b) and purified. It has been found that compounds of Formula IV can be easily isolated by precipitation. Compounds of Formula IV can be precipitated by adding water to the reaction mixture after the halogenation reaction is complete. Advantageously, the mixture is kept at room temperature for a few hours, such as about 1-2 hours. The isolation can be continued by adding more water and agitating the mixture for an additional 1-2 hours, and using filtration to collect the precipitate. It has been found that compounds of Formula IV can be purified by washing the precipitate obtained from filtration with a suitable solvent or mixtures thereof. Suitable solvents for washing the precipitate include water, methanol, acetone, THF, isopropyl ether, n-heptane, or mixtures thereof. Advantageously, the solvent is isopropyl ether, n-heptane or a mixture of acetone and heptane. Preferably, the solvent is 5% acetone in n-heptane. After washing, the precipitate can be dried in a vacuum oven at low temperature, such as below 50° C. A purity of >99% (area percent, determined by HPLC) can be obtained by washing the precipitate. Compounds of Formula IV can also be purified by recrystallization. Advantageously, the precipitate is recrystallized from a solvent listed above for washing or mixtures thereof.

In a further aspect, the multistep process of preparing 3α-hydroxy-3β-alkoxymethyl-21-substituted-pregnan-20-ones of Formula I comprises the step of

(c) reacting the product mixture of step (b) comprising a compound of Formula IV or reacting an isolated compound of Formula IV with a nitrogen-containing, optionally substituted heteroaryl compound or an alkali metal salt of a nitrogen-containing, optionally substituted heteroaryl compound to form a compound of Formula I.

Suitable nitrogen-containing, optionally substituted heteroaryl compounds for use in step (c) include, but are not limited to, oxazole, thiazole, tetrazole, imidazole, pyrrole, pyridine, pyrimidine, quinoline, and isoquinoline, each of which is optionally substituted. Preferred nitrogen-containing, optionally substituted heteroaryl compounds for use in step (c) include imidazole and tetrazole, where imidazole is most preferred.

Optional substituents on the nitrogen-containing heteroaryl ring include one or more of halo, haloalkyl, aryl, heterocyclo, cycloalkyl, heteroaryl, alkyl, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, aminoalkyl, carboxyalkyl, alkoxyalkyl, nitro, amino, ureido, cyano, acylamino, hydroxy, thiol, acyloxy, azido, alkoxy, carboxy, aminocarbonyl, and alkylthiol. Preferred optional substituents include halo, halo(C₁₋₆)alkyl, hydroxy(C₁₋₆)alkyl, amino(C₁₋₆)alkyl, nitro, C₁₋₆ alkyl, C₁₋₆ alkoxy and amino, more preferably halo, halo(C₁₋₃)alkyl, hydroxy(C₁₋₃)alkyl, amino(C₁₋₃)alkyl, nitro, C₁₋₃ alkyl, C₁₋₃ alkoxy and amino, more preferably C₁₋₃ alkyl or C₁₋₃ alkoxy. One or more optional substituents can be attached to carbon atoms and/or nitrogen atoms of the nitrogen-containing heteroaryl ring. Suitably, the nitrogen-containing heteroaryl group includes 0, 1, 2, or 3 optional substituents, preferably 0, 1, or 2 optional substituents.

In a preferred embodiment, compounds of Formula IV are reacted with an alkali metal salt of a nitrogen-containing, optionally substituted heteroaryl compound in the reaction step (c). It has been found that by using an alkali metal salt of a nitrogen-containing, optionally substituted heteroaryl compound in step (c), the number and amount of by-products are reduced and the yield of the 17β isomer is increased. Suitable alkali metal salts include lithium, sodium and potassium salts, where lithium salts are preferred. A lithium salt of a heteroaryl compound can be prepared by reacting the heteroaryl with, e.g., LiH, LiOH, or LiOH×H₂O. Preferably, the lithium salt is prepared by reacting the heteroaryl, such as imidazole, with LiH. A sodium salt of a heteroaryl compound can be prepared by reacting the heteroaryl with, e.g., NaH, NaOH, or NaOMe. A potassium salt of a heteroaryl compound can be prepared by reacting the heteroaryl with, e.g., t-BuOK. It has been found that the yield of 17α isomer is increased in strongly basic conditions provided by Na-imidazole or K-imidazole. The lithium salt is preferred when a higher yield of 17β isomer of compound of Formula I is desired.

Advantageously, the reaction of step (c) is conducted using Li-imidazole as the reagent in a suitable solvent, e.g., THF, at about −20° C. to about +10° C., preferably at about −10° C. to about +10° C. Advantageously, the reaction temperature is maintained at −10° C. The reaction time is suitably from about 15 minutes to about 1 hour, preferably from about 0.5 hour to about 1 hour at about 0° C. to about 10° C. Advantageously, the reaction time is about 0.5 hour at −10° C. Preferably, the starting compound of Formula IV is of more than 80% (area percent, determined by HPLC) purity. Advantageously, the reaction is conducted as depicted in Scheme 1 as follows:

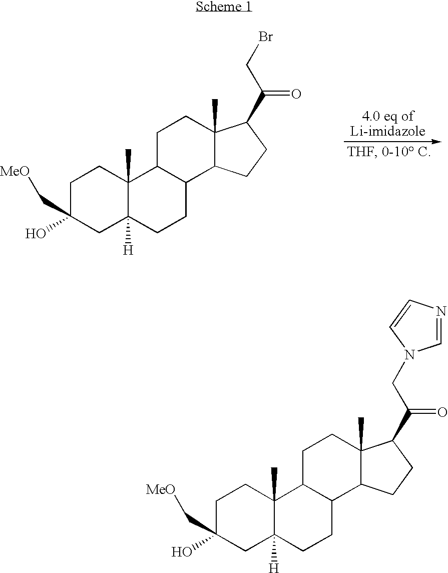

After the reaction of step (c), the reaction is quenched and compounds of Formula I are isolated from the reaction mixture and purified. The reaction can be quenched, e.g., by treating the reaction mixture with aqueous ammonium chloride solution, water and aqueous saturated sodium chloride solution. Advantageously, the reaction is quenched with aqueous NH₄Cl/NaCl solution for about 30 minutes. The compound of Formula I can be isolated from the reaction mixture by conventional methods, such as by extracting into a suitable solvent, concentrating the organic phase by, e.g., distillation, and precipitating the crude product from a suitable solvent, such as ethyl acetate, n-heptane or mixtures thereof. The crude product of Formula I can be further purified by hot filtration. Accordingly, the crude product is suspended into a suitable solvent, the suspension is heated to reflux temperature, aluminium sulfate is added, and the mixture is filtered hot. The filtrate is concentrated and more pure product is precipitated. By the above steps, about 85% (w/w) purity of the product can be achieved. In order to obtain pure compound of Formula I, the product from the hot filtration above can be further purified chromatographically, e.g., by flash chromatography as described in Example 6 (f) below.

It has been found that a tedious purification process of the compound of Formula I can be avoided by using a lithium or a sodium salt of a heteroaryl compound in reaction step (c) as described above. Advantageously, a lithium salt of the nitrogen-containing, optionally substituted heteroaryl compound is used. After the reaction with a lithium or sodium salt, the crude product can be isolated from the reaction mixture by precipitating with a suitable solvent, such as toluene. The crude product of Formula I can be purified by recrystallization from a suitable solvent, such as methanol, isopropylether, or acetone or mixtures thereof. Advantageously, the product of Formula I is recrystallized from a methanol/isopropylether solution.

Compounds of Formula II can be prepared by reacting a compound of Formula V:

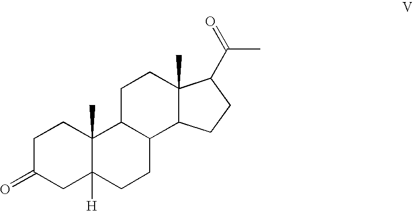

with a reactant capable of selectively forming an oxirane ring at the 3-position keto group of the compound of Formula V. Preferably, compounds of Formula V are primarily 5α and 17β isomers. A preferred reactant capable of selectively forming an oxirane ring is an ylide compound. The ylide can be obtained by mixing a Corey's reagent and a base in a suitable aprotic polar solvent to form an ylide (Corey et al., J. Am. Chem. Soc. 87:1354-1364, 1965). The ylide is mixed with the compound of Formula V, which has been suspended or dissolved in an appropriate solvent. Sufficient reagent is provided to produce an amount of ylide that will give complete reaction of the ketone. The amount of the base used to produce the ylide should be chosen so as to leave no unreacted base after the formation of the ylide. The reaction is performed in a dry atmosphere, such as under nitrogen or argon gas, with dry solvents (in the absence of water). The time and temperature of the ylide formation and the subsequent reaction with the ketone can be determined by monitoring loss of the ketone starting material or the formation of the oxirane product of Formula II using a suitable analytical technique, such as TLC or HPLC.

Advantageously, the base, Corey's reagent and solvent are first mixed at an elevated temperature, e.g., at about 50 to about 70° C., preferably at about 65° C., for about 1-3 hours, preferably about 2 hours, and then cooled to room temperature. Compound of Formula V is then added to the reaction mixture and the temperature is maintained at from about 25° C. to about 35° C. After the reaction is complete, water is added to precipitate the product. Advantageously, the product is purified by, e.g., recrystallization to minimize the amount of by-products in the next reaction step (a). Suitable solvents for re-crystallizing compounds of Formula II include polar or weakly polar solvents or mixtures thereof, such as methanol, acetone, ethyl acetate, isopropyl alcohol or mixtures thereof, especially acetone:methanol or ethyl acetate:methanol from 1:3 to 1:5 (v:v), and preferably 1:3 (v:v). Advantageously, the purity of the compound of Formula II is at least 85%, preferably at least 95% (area percent) as determined by HPLC. The HPLC conditions are as follows: Phenomenex, Luna C₁₈₍₂₎, 3 μm, 15 cm (L)×4.6 mm (ID) column or equivalent; column temperature 30° C.; refractive index (RI) detector; detector temperature 30° C.; flow rate 0.8 mL/min; and mobile phase 80% methanol (v/v).

The reagent can be any Corey's reagent that reacts chemoselectively so as to selectively convert only the 3-keto group to an oxirane. The reagent is also chosen for the ability to diastereoselectively convert the 3-keto group to the desired oxirane, in this case a 3(R)-pregnan-3-spiro-2′oxirane-20-one. Preferably, the reagent is trimethylsulfoxonium iodide (Me₃SOI), but any equivalent reagent that will react with the appropriate selectivity will suffice.

The Corey's reagent is dissolved in an appropriate aprotic, polar solvent, such as a polyether, an amide, a phosphoric amide, a sulfoxide, a sulfolane, or mixtures thereof. Suitable solvents include dimethylsulfoxide (DMSO), tetrahydrofuran (THF), hexamethylphosphoric triamide (HMPT), sulfolane, N-methyl-pyrrolidone, dioxane, dimethoxyethane (DME), and dimethylformamide (DMF).

The basicity of the base is sufficiently high to remove a proton from Corey's reagent in order to form the ylide. Suitable bases include NaH, potassium t-butoxide, and NaNH₂.

Compounds of Formula V can be prepared by allowing a compound of Formula VI:

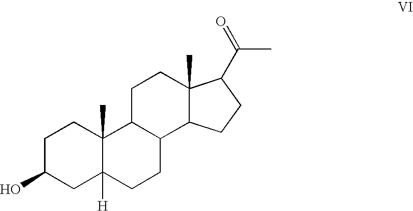

to react with an oxidizing agent. Preferably, the compound of Formula VI is primarily 5α and 17β isomer. Suitable oxidation agents include, but are not limited to, alkali metal hypohalides, e.g., NaOCl and LiOBr. Advantageously, NaOCl is used.

The compound of Formula VI can be prepared by reacting pregnenolone, i.e., a compound of Formula VII:

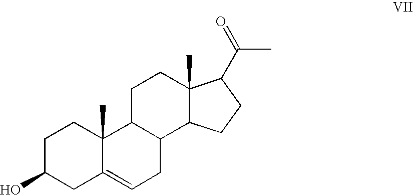

with hydrogen in a suitable solvent in the presence of a hydrogenation catalyst. The compound of Formula VII is primarily 17β isomer. Suitable hydrogenation catalysts include, but are not limited to, palladium-on-carbon, palladium-on-barium sulfate (Alfa Aesar, Ward Hill, Mass.), platinum-on-barium sulfate (Engelhard, Iselin, N.J.), and rhodium-on-barium sulfate (Engelhard).

Preferably, such hydrogenation conditions are used that produce primarily a isomers at the 5-position hydrogen of the steroid ring system. Primarily the 5α isomer is produced when pregnenolone is reacted with hydrogen in the presence of palladium-on-carbon. Advantageously, 5-20% palladium-on-carbon is used. Pregnenolone is commercially available.

Preferred compounds of Formula I that can be synthesized according to the present invention include without limitation:
3α-hydroxy-21-(1′-imidazolyl)-3β-methoxymethyl-5α-pregnan-20-one; and
3α-hydroxy-3β-methoxymethyl-21-(2′-tetrazolyl)-5α-pregnan-20-one.

The most preferred embodiment of the present invention is the process for preparing 3α-hydroxy-21-(1′-imidazolyl)-3β-methoxymethyl-5α-pregnan-20-one (6) having the formula:

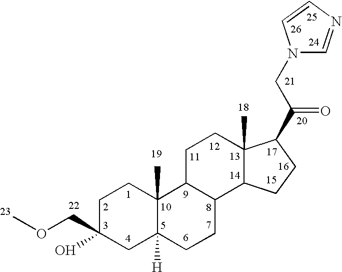

In one aspect, the present invention relates to a process, comprising reacting 5(3R)-spiro[oxirane-2′,5α-pregnan]-20-one (3) of formula:

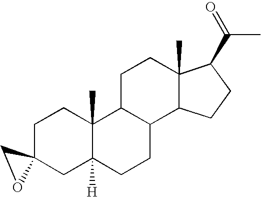

with NaOMe or NaOH in an appropriate solvent to obtain a mixture comprising 3α-hydroxy-3β-methoxymethyl-5α-pregnan-20-one (4) of the formula

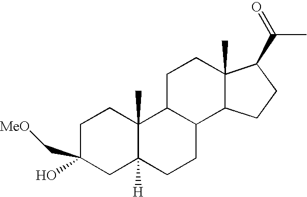

Preferably, the solvent is methanol. Preferably, the reaction temperature is about 35° C. to about 45° C. when 5(3R)-spiro[oxirane-2′,5α-pregnan]-20-one is reacted with NaOH in methanol. Preferably, the reaction temperature is reflux temperature when 5(3R)-spiro[oxirane-2′,5α-pregnan]-20-one is reacted with NaOMe in methanol. Preferably, the 17β isomer is isolated and purified from the mixture before using it in the next step.

The present invention also relates to a process, comprising reacting 3α-hydroxy-3β-methoxymethyl-5α-pregnan-20-one (4) of the formula

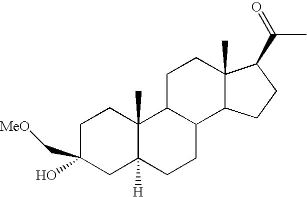

with Br₂ in the presence of HBr to obtain a mixture comprising a compound 21-bromo-3α-hydroxy-3β-methoxymethyl-5α-pregnan-20-one (5) of formula

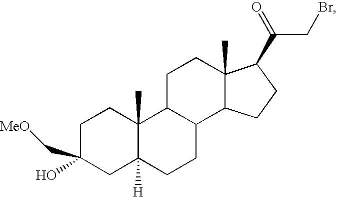

then isolating the compound from the mixture, and optionally purifying the compound.

In a further aspect, the present invention relates to a process for preparing 3α-hydroxy-21-(1′-imidazolyl)-3β-methoxymethyl-5α-pregnan-20-one (6), comprising reacting 21-bromo-3α-hydroxy-3β-methoxymethyl-5α-pregnan-20-one (5) of formula

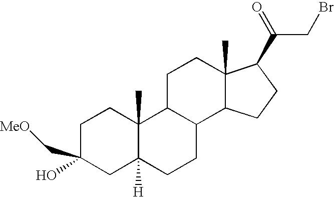

with Li-imidazole to obtain a mixture comprising the product, 3α-hydroxy-21-(1′-imidazolyl)-3β-methoxymethyl-5α-pregnan-20-one (6). The product can be isolated from the reaction mixture by precipitating with a suitable solvent, such as toluene, to obtain a crude product. The reaction is preferably quenched with aqueous NH₄Cl/NaCl solution, preferably for about 30 minutes, before the precipitation. The crude product can be re-crystallized from a suitable solvent, such as methanol, isopropylether, or acetone or mixtures thereof. Advantageously, the product is re-crystallized from methanol/isopropylether solvent.

It has been found that some of the by-products formed during the synthesis of 3α-hydroxy-3β-alkoxymethyl-pregnan-20-ones are novel compounds that have GABA_{A} receptor binding activity. Accordingly, one aspect of the present invention is a 3α-hydroxy-3β-alkylthioethyl-pregnan-20-one compound of Formula VIII

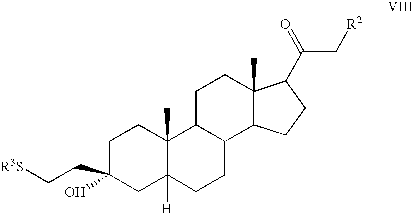

or a pharmaceutically acceptable salt, prodrug or solvate thereof, wherein R² is as defined above and R³ is an alkyl group, preferably C₁₋₆ alkyl, more preferably C₁₋₄ alkyl, and especially methyl. These thio-compounds of Formula VIII can be prepared by isolating them from the reaction mixture after step (c). Alternatively they can be prepared, e.g., by reacting brominated 3α-hydroxy-3β-alkylthioethyl-pregnan-20-ones, prepared as described in Scheme 2 below, with an appropriate optionally substituted heteroaryl compound or a salt thereof as described herein.

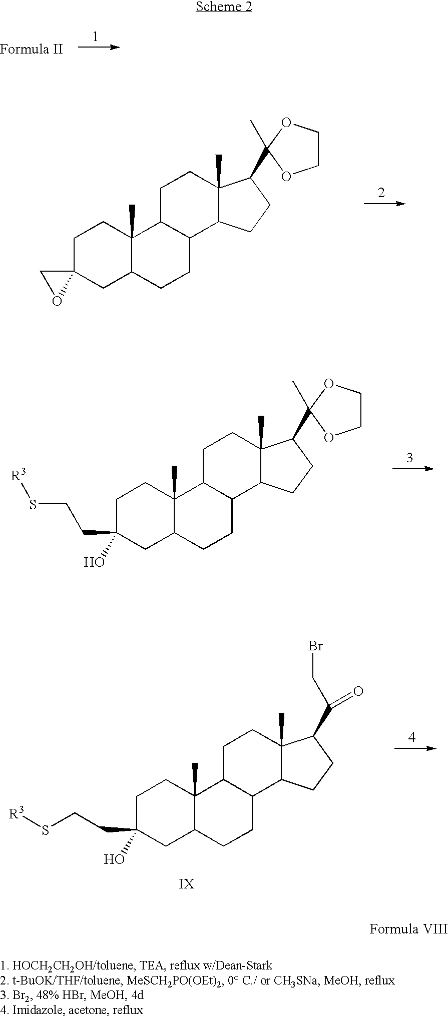

Advantageously, compounds of Formula IX are reacted with imidazole in acetone at reflux temperature to obtain compounds of Formula VIII. Preferably, R³ is methyl. Preferably, compounds of Formula VIII are 5α and 17β isomers. Advantageously, the compound is 3α-hydroxy-21-(1′-imidazolyl)-3β-methylthioethyl-5α-pregnan-20-one (11) having the formula

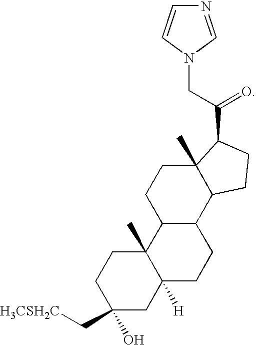

The present invention is also directed to the 17α isomers of compounds of Formula I, i.e., to a compound of the following Formula X

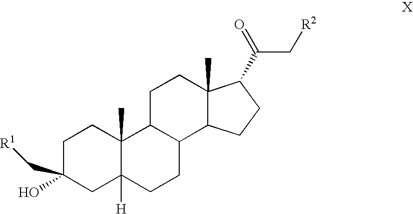

or a pharmaceutically acceptable salt, prodrug or solvate thereof, wherein R¹ and R² are as defined above. Compounds of Formula X can be prepared by methods known in the art or as described herein. Preferably, the compound of Formula X is the 5α isomer. A preferred compound of Formula X is 17α-acetyl-3α-hydroxy-21-(1′-imidazolyl)-3β-methoxymethyl-5α-pregnan-20-one (7).

Further, the present invention is directed to compounds of Formula XI

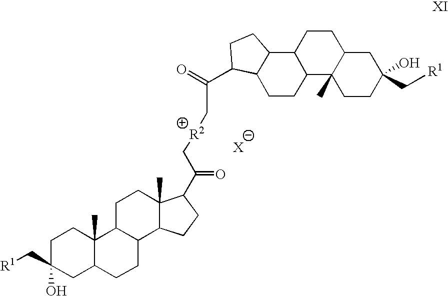

or a pharmaceutically acceptable salt, prodrug or solvate thereof, wherein each R¹ is independently selected as defined above, and R² is as defined above and X is a halogen, preferably bromine, provided that R² is an optionally substituted, N-attached heteroaryl group having at least two nitrogen atoms wherein each of the two nitrogen atoms is substituted with one of the tails of Formula XI. Preferably, R² is imidazolyl or tetrazolyl. Preferably, compounds of Formula XI are 5α and 17β isomers. Compounds of Formula XI can be prepared, e.g., by allowing compounds of Formula IV to react with compounds of Formula I as described in Example 8 below. Preferably, the compound of Formula XI is a 2,3-dihydro-1,3-di(3α-hydroxy-3β-methoxymethyl-5α-pregnan-20-one-21-yl)-imidazonium salt of the formula

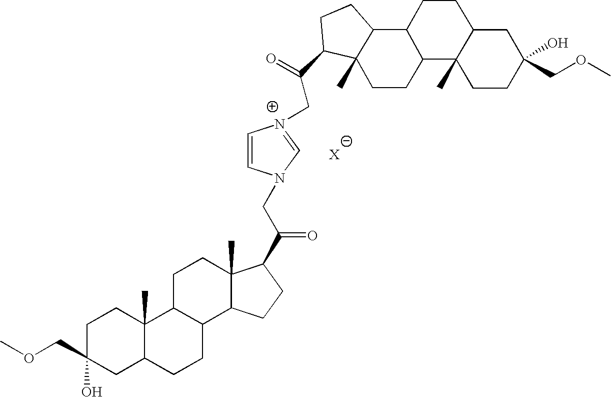

wherein X is a halogen, preferably bromine (10).

It has also been found that certain metabolites of 17β-acetyl-3α-hydroxy-21-(1′-imidazolyl)-3β-methoxymethyl-5α-pregnan-20-one have GABA_{A} receptor binding activity. Accordingly, the present invention is directed to compounds of Formulae XII and XIII as follows:

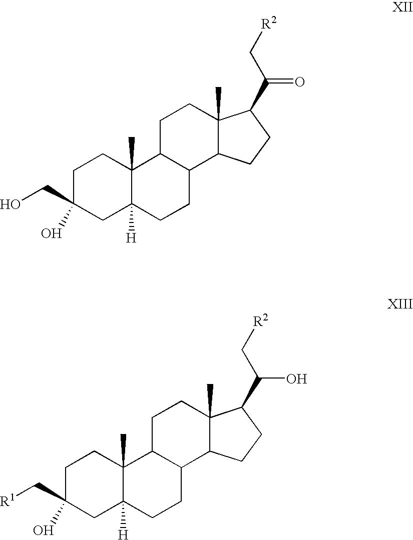

or pharmaceutically acceptable salts or solvates thereof, wherein R¹ and R² are as defined above. Preferably, R¹ is C₁₋₆ alkyl, more preferably C₁₋₄ alkyl, and especially methyl. Preferably, R² is an N-attached, optionally substituted heteroaryl selected from the group consisting of oxazolyl, thiazolyl, tetrazolyl, imidazolyl, pyrrolyl, pyridyl, pyrimidyl, quinolinyl, and isoquinolinyl, more preferably imidazolyl or tetrazolyl, and especially imidazolyl. Preferably, the compound exists in a composition that includes at least 2% of the compound by weight, preferably at least 5% by weight. Preferred compounds of Formulae XII and XIII include the following compounds:

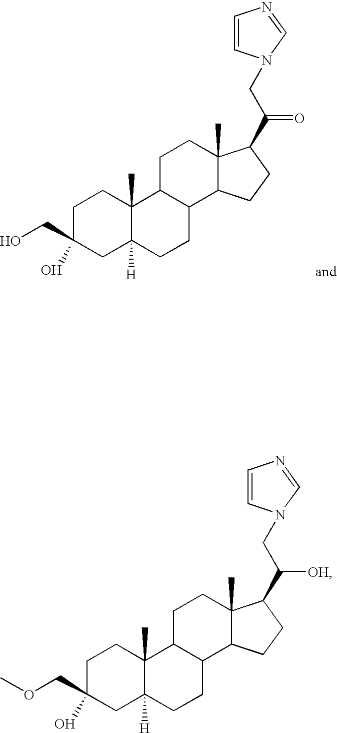

and
respectively.

Compounds of Formula XII can be prepared by the method described in Example 10 below. Accordingly, a compound of Formula II is reacted with sodium dissolved in benzyl alcohol to open the oxirane ring, the 21-position is brominated, and the brominated compound is reacted with an appropriate heteroaryl compound. The benzyl protection is removed to obtain compounds of Formula XII.

Compounds of Formula XIII can be prepared by the method described in Example 11 below. Accordingly, compounds of Formula XIII can be prepared by reduction of the keto group at the 20-position of 17β compounds of Formula I to obtain the corresponding hydroxy derivatives of Formula XIII.

Certain of the compounds of Formulae VIII, X, and XI may exist as optical isomers and the invention includes both the racemic mixtures of such optical isomers as well as the individual enantiomers that may be separated according to methods that are well known to those of ordinary skill in the art.

Also included within the scope of the present invention are the non-toxic pharmaceutically acceptable salts of the compounds of the present invention. Examples of pharmaceutically acceptable addition salts include inorganic and organic acid addition salts such as hydrochloride, hydrobromide, phosphate, sulphate, citrate, lactate, tartrate, maleate, fumarate, mandelate, acetate, dichloroacetate, and oxalate. Acid addition salts are formed by mixing a solution of the particular heteroaryl compound of the present invention with a solution of a pharmaceutically acceptable non-toxic inorganic or organic acid acid such as hydrochloric acid, fumaric acid, maleic acid, succinic acid, acetic acid, citric acid, tartaric acid, carbonic acid, phosphoric acid, oxalic acid, dichloroacetic acid, and the like.

The invention disclosed herein is also meant to encompass prodrugs of the compounds of Formulae VIII, X, and XI. Prodrugs are considered to be any covalently bonded carriers which release the active parent drug in vivo. Examples of prodrugs include esters or amides of the compounds of Formulae VIII, X, and XI with optional substitution including hydroxyalkyl or aminoalkyl, and these may be prepared by reacting such compounds with anhydrides such as succinic anhydride.

Useful aryl groups are C₆₋₁₄ aryl, especially C₆₋₁₀ aryl. Typical C₆₋₁₄ aryl groups include phenyl, naphthyl, phenanthryl, anthracyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful halo or halogen groups include fluorine, chlorine, bromine and iodine.

Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups.

Useful alkenyl groups are C₂₋₆ alkenyl groups, preferably C₂₋₄ alkenyl. Typical C₂₋₄ alkenyl groups include ethenyl, propenyl, isopropenyl, butenyl, and sec-butenyl.

Useful alkynyl groups are C₂₋₆ alkynyl groups, preferably C₂₋₄ alkynyl. Typical C₂₋₄ alkynyl groups include ethynyl, propynyl, butynyl, and 2-butynyl groups.

Useful arylalkyl groups include any of the above-mentioned C₁₋₁₀ alkyl groups substituted by any of the above-mentioned C₆₋₁₄ aryl groups. Useful values include benzyl, phenethyl and naphthylmethyl.

Useful arylalkenyl groups include any of the above-mentioned C₂₋₄ alkenyl groups substituted by any of the above-mentioned C₆₋₁₄ aryl groups.

Useful arylalkynyl groups include any of the above-mentioned C₂₋₄ alkynyl groups substituted by any of the above-mentioned C₆₋₁₄ aryl groups. Useful values include phenylethynyl and phenylpropynyl.

Useful cycloalkylalkyl groups include any of the above-mentioned C₁₋₁₀ alkyl groups substituted by any of the above-mentioned cycloalkyl groups.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl and trichloromethyl groups.

Useful hydroxyalkyl groups include C₁₋₁₀ alkyl groups substituted by hydroxy, e.g. hydroxymethyl, hydroxyethyl, hydroxypropyl and hydroxybutyl groups.

Useful alkoxy groups include oxygen substituted by one of the C₁₋₁₀ alkyl groups mentioned above.

Useful alkylthio groups include sulfur substituted by one of the C₁₋₁₀ alkyl groups mentioned above.

Useful acylamino groups are any acyl group, particularly C₂₋₆ alkanoyl or C₆₋₁₀ aryl(C₂₋₆)alkanoyl attached to an amino nitrogen, e.g. acetamido, propionamido, butanoylamido, pentanoylamido, hexanoylamido, and benzoyl.

Useful acyloxy groups are any C₁₋₆ acyl(alkanoyl) attached to an oxy (—O—) group, e.g. acetoxy, propionoyloxy, butanoyloxy, pentanoyloxy, hexanoyloxy and the like.

The term heterocyclic is used herein to mean saturated or wholly or partially unsaturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable. Examples include, but are not limited to, pyrrolidine, piperidine, piperazine, morpholine, imidazoline, pyrazolidine, benzodiazepines, and the like.

Useful heterocycloalkyl groups include any of the above-mentioned C₁₋₁₀ alkyl groups substituted by any of the above-mentioned heterocyclic groups.

Useful heteroaryl groups include any of the following: thienyl, benzo[b]thienyl, thainhrenyl, furyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, quinozalinyl, cinnolinyl, isothiazolinyl, phenothiazinyl, isoxazolyl, furazanyl, phenoxazinyl, 1,4-dihydroquinaxaline-2,3-dione, 7-aminoisocoumarin, pyrido[1,2-a]pyrimidin-4-one, 1,2-benzoisoxazol-3-yl, 4-nitrobenzofurazan, benzimidazolyl, 2-oxindolyl, and 2-oxobenzimidazolyl.

Useful heteroarylalkyl groups include any of the above-mentioned C₁₋₁₀ alkyl groups substituted by any of the above-mentioned heteroaryl groups.

Useful heteroarylalkenyl groups include any of the above-mentioned C₂₋₆ alkenyl groups substituted by any of the above-mentioned heteroaryl groups.

Useful heteroarylalkynyl groups include any of the above-mentioned C₂₋₆ alkynyl groups substituted by any of the above-mentioned heteroaryl groups.

Aminocarbonyl group is —C(O)NH₂.

Useful alkylthiol groups include any of the above-mentioned C₁₋₁₀ alkyl groups substituted by a —SH group.

A carboxy group is —COOH.

An azido group is —N₃.

An ureido group is —NH—C(O)—NH₂.

An amino group is —NH₂.

Compounds of the present invention may be tested for their GABA_{A} binding activity by the following in vitro binding assay.

It is known for those skilled in the art that [³⁵S]t-butylbicyclophosphorothionate ([³⁵S]TBPS) is a ligand of the GABA receptor that binds to the channel region of the receptor complex. Neuroactive steroids allosterically inhibit the binding of [³⁵S]TBPS. [³⁵S]TBPS binding assays were conducted by sequentially mixing the following reagents in a 96-deep well polypropylene plates (Costar) in the order shown to yield the indicated final concentrations: 100 μL GABA (200 μM; Sigma; 20 μM final), 100 μL membrane protein (prepared from HEK293 cells expressing GABA_{A} subunits α1, β2, and γ2) (25 μg/mL final), 5 μL of a 200× stock solution of a compound dilution series (final 10 μM to 0.6 μM) prepared in dimethylsulfoxide (DMSO) or 400 μM TBPS (final 2 μM) (non-specific binding) to 754 μL binding buffer (50 mM Na—K phosphate/200 mM NaCl). The prepared membrane solution (1000 μL/well) was transferred to 96-deep well polypropylene plates (Costar) containing 5 μL of 2 mM stock solution of compound or appropriate control prepared in dimethylsulfoxide (DMSO) (total binding) or 400 μM TBPS (non-specific binding). 3α-5α-pregnalone served as the assay positive control. Plates were incubated for 90 minutes at room temperature with shaking. Reactions were terminated by rapid filtration onto 96-well Unifilter GF/B filter plates (Packard) using a 96-well tissue harvester (Filtemate, Packard) and followed by 4 filtration washes with 1 mL of ice-cold binding buffer. Filter plates were subsequently dried at 50 EC for several hours. The bottoms of the dried plates were sealed and 50 μL/well scintillation cocktail was added and plates were counted in a Packard Top-Count for 1 min/well. The compounds were tested in a 12-point half-log dilution dose course, starting at 10 μM. Each dose point was measured in duplicate. 3α-5α-pregnalone was tested in singlet on each plate. Triplicate wells of total binding and non-specific binding were also included on each plate.

For each dose, a percent inhibition based on the total binding (TB) and non-specific binding (NSB) was calculated using the equation:

% inhibition=100×(1−(average sample−NSB)/(TB−NSB)) Eq.1

Percent inhibition values were plotted against compound concentration and the resulting curve was analyzed using XIFit3 (IDBS). The IC₅₀ values were calculated from the curve. A value of “No Fit” was reported if the curve did not reflect a classic binding curve. The IC₅₀ values were represented as mean±S.E.M.

Compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for insomnia. For intramuscular injection, the dose is generally about one-half of the oral dose.

The unit oral dose may comprise from about 0.01 to about 50 mg, and preferably from about 0.1 to about 10 mg of the compound. The unit dose may be administered one or more times daily as one or more tablets each containing from about 0.1 to about 100, conveniently about 0.25 to 50 mg of the compound or its salt or solvate.

In addition to administering the compound as a raw chemical, the compounds of the invention may be administered as part of a pharmaceutical preparation containing one or more suitable pharmaceutically acceptable carriers comprising excipients and auxiliaries which facilitate processing of the compounds into preparations which can be used pharmaceutically. Preferably, the preparations, particularly those preparations which can be administered orally and which can be used for the preferred type of administration, such as tablets, dragees, and capsules, and also preparations which can be administered rectally, such as suppositories, as well as suitable solutions for administration by injection or orally, contain from about 0.01 to about 99 weight percent, preferably from about 0.25 to about 75 weight percent of active compound(s), together with the excipient.

The pharmaceutical composition of the invention may be administered to any animal that can experience a beneficial effect of a compound of the invention. Foremost among such animals are mammals, e.g., humans, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment if any, frequency of treatment, and the nature of the effect desired.

The pharmaceutical preparations of the present invention are manufactured in a standard manner, for example, by means of conventional mixing, granulating, dragee-making, dissolving, or lyophilizing processes. Thus, pharmaceutical preparations for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example, tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate. Auxiliaries include flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which can be used rectally, include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances which increase the viscosity of the suspension, and include, for example, sodium carboxymethyl cellulose, sorbitol, and/or dextran. Optionally, the suspension may also contain stabilizers.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

The yields of the following examples were not optimized, and MS spectra for all of the compounds were obtained with LCMS. The reactions were followed by either TLC or/and LCMS or/and ¹H NMR. The purity percents given are area percents determined by HPLC if not specified otherwise.

### EXAMPLE 1

### 3β-Hydroxy-5α-pregnan-20-one (1)

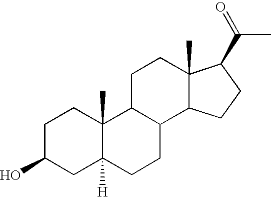

A solution of pregnenolone (Changzhou Medical Raw Material Factory, Zhenglu Town, Changzhou City, Jiangsu, China) (100 g, 0.316 mol) dissolved in THF/toluene (1:1, 1.0 L) was treated with a suspension of 10% Pd/C (10 g) in 200 mL of 1:1 THF/toluene. Glacial acetic acid (67 mL) was added to the reaction mixture and the resulting mixture was placed in a stirred autoclave at 60° C. under 60 psi of hydrogen gas. After about 18 to about 24 hours, the reaction was monitored by HPLC and by ¹H NMR for completion. The reaction mixture was filtered through a CELITE (diatomaceous earth) bed. The CELITE and the reactor were washed with acetone (500 mL and 2 L, respectively). The organic phase was concentrated under reduced pressure to give the intermediate 3β-hydroxy-5α-pregnan-20-one (1) (85.5 g, 85% yield) having mp 194-195° C., which contains a small amount of a dehydroxyl side product.

### EXAMPLE 2

### 5α-Pregnan-3,20-dione (2)

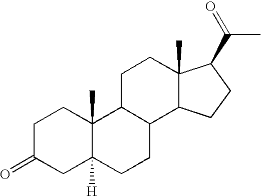

A 5.0 L reaction vessel was charged with 3β-hydroxy-5α-pregnan-20-one (1) (126.9 g, 0.398 mol) dissolved in 2.2 L glacial acetic acid. Sodium bromide (4.09 g, 0.0398 mol) was dissolved in a 12.0% solution of aqueous NaOCl (395 mL, 47.4 g, 0.637 mol) and the mixture was added dropwise into the reaction vessel. The reaction temperature was kept at from about 28 to about 35° C., and the biphasic mixture was stirred rapidly for 4-5 hours. HPLC or TLC (3:7 ethyl acetate/hexane) was used to monitor the reaction until the 3β-hydroxy-5α-pregnan-20-one (1) was completely consumed, and the less polar product 5α-pregnan-3,20-dione (2) was formed. If the 3β-hydroxy-5α-pregnan-20-one (1) was not completely consumed, 0.2 equivalents of 12.0% solution of NaOCl (0.0796 mol, 5.93 g, 49.4 mL) was charged one or more times to the reaction mixture until the quantity of 3β-hydroxy-5α-pregnan-20-one (1) was less than a 5% ratio in total yield determined by HPLC. Water (1.5 L) was added, and the solid precipitate that was formed was dissolved in toluene (1.5 L). The organic layer was washed with water (1 L×2), 5% Na₂SO₃ aqueous solution (500 mL×2), a saturated aqueous NaHCO₃ solution (1.0 L×2), and brine (1.0 L×2) to give the intermediate 5α-pregnan-3,20-dione (2) in toluene solution.

### EXAMPLE 3

### 5(3R)-Spiro[oxirane-2′,5α-pregnan]-20-one (3)

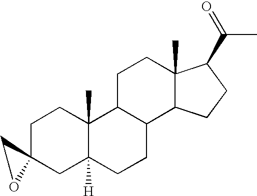

Potassium tert-butoxide (133.9 g, 1.19 mol) was added to a stirred solution of trimethylsulfoxonium iodide (262.7 g, 1.19 mol) in 600 mL of THF and 400 mL of DMSO under N₂. After stirring at room temperature for 30 minutes to 1 hour, the solution of 5α-pregnan-3,20-dione (2) (0.398 mol, 125.9 g) in 2.0 L of toluene was added dropwise via addition funnel. The reaction temperature was kept at from about 35° C. to about 45° C. After 1 hour, the reaction was monitored by HPLC or TLC (3:7 ethyl acetate/hexanes) which indicated a complete consumption of 5α-pregnan-3,20-dione (2) and the formation of the less polar product 5(3R)-spiro[oxirane-2′,5α-pregnan]-20-one (3). Water (1.0 L) was added, and the solid precipitate was dissolved in ethyl acetate (1.0 L). The organic layer was separated, washed with H₂O (1.0 L×2) and brine (1.0 L×2), and evaporated to dryness. The light yellow solid product (3) was afforded and washed with MeOH (150 mL×2) until the amount of compound (2) was less than 2% as measured by HPLC method. A white solid was obtained (85.0 g) in about 64% yield.

### EXAMPLE 4

### 3α-Hydroxy-3β-methoxymethyl-5α-pregnan-20-one (4)

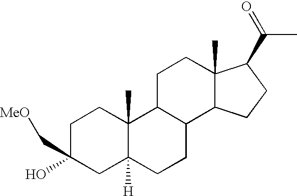

Solid sodium methoxide (27.5 g, 0.192 mol) was added to a solution of 5(3R)-spiro[oxirane-2′,5α-pregnan]-20-one (3) (84.0 g, 0.254 mol) in 1.0 L of MeOH under N₂. The reaction mixture was heated to reflux for 4-5 hours. The reaction was monitored by HPLC or TLC (3:7 ethyl acetate/hexanes) which indicated a complete consumption of the oxirane (3) and the formation of the more polar product 3α-hydroxy-3β-methoxymethyl-5α-pregnan-20-one (4). Once the reaction mixture reached room temperature, 10 mL of glacial acetic acid was added dropwise and the reaction mixture was concentrated in vacuo to approximately 250 mL. Water (1.0 L) was then added and a solid precipitate was formed. The solid precipitate was filtered to give a white solid product (4) in 84% yield (77.2 g, the purity was more than 95%, area percent determined by HPLC). The product was approximately a 9:1 ratio of the 17β:17α-acetyl epimers, as determined by ¹H NMR, and it was used immediately in the following example as such.

### EXAMPLE 5

3α-Hydroxy-21-(1′-imidazolyl)-3β-methoxymethyl-5α-pregnan-20-one (6)

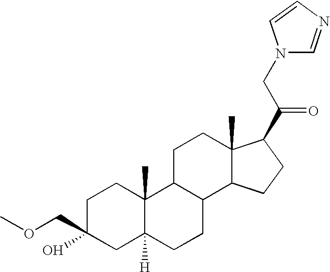

### (a) 21-Bromo-3α-hydroxy-3β-methoxymethyl-5α-pregnan-20-one (5)

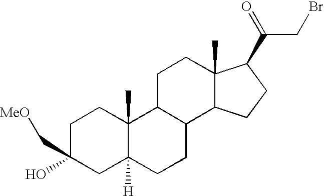

3 drops of a 48% aqueous HBr solution were added to a solution of 3α-hydroxy-3β-methoxymethyl-5α-pregnan-20-one (4) (prepared in Example 4) (10.0 g, 0.0276 mol) in 200 mL of MeOH while stirring at 0° C. Bromine (4.63 g, 0.0290 mol) was then added dropwise as a solution in 100 mL of MeOH over 1.5 hours during which the reaction was shielded from light. After an additional 30 minutes, TLC (1% acetone/dichloromethane) analysis indicated the consumption of starting material and the formation of the less polar product 21-bromo-3α-hydroxy-3β-methoxymethyl-5α-pregnan-20-one (5). The reaction mixture was concentrated to approximately 100 mL and dichloromethane (300 mL) was then added. The organic layer was washed with water (50 mL×2) and then with brine (100 mL×2), and concentrated affording compound (5) as a pale yellow solution. No further purification was carried out. The product was used immediately in the next step.

### (b) 3α-Hydroxy-21-(1′-imidazolyl)-3β-methoxymethyl-5α-pregnan-20-one (6)

Imidazole (9.4 g, 0.138 mol) was added to a solution of compound (5) in 200 mL of acetone and the reaction mixture was heated to reflux under N₂. The reaction was completed after 2-3 hours as determined by TLC (95:4.5:0.5 CH₂Cl₂:MeOH:triethylamine (TEA)). The reaction mixture was washed with water (50 mL×2) and then concentrated under reduced pressure to give the resulting oil. Following a solubility study of the final product (6) and the intermediate (5) in different solvents, ethyl acetate and hexane were used to recrystallize the final product. Accordingly, the resulting oil was mixed with a solution of 30 mL of ethyl acetate and 90 mL of hexanes, from which the crude compound (6) precipitated. The mixture was filtered and the filtrate was collected and dried in a vacuum oven to yield an off-white solid product. The crude product was then washed with ethyl acetate to give compound (6) (7.2 g, 0.0691 mol, 61% yield from compound (4)) having about 85% purity as determined by ¹H NMR. The product was further purified by passing it through a short silica gel column. The pure product was obtained as a white solid in 43% yield, mp 185-187° C. (evacuated capillary), and the chemical structure of the product was confirmed by NMR, IR, and MS. A sample of this material was analyzed by reverse-phase HPLC and the result indicated >98% (area percent) purity. ¹H NMR (400 MHz, DMSO-d₆): δ 0.56 (s, 3H), 0.66 (s, 3H), 0.72 (m, 1H), 0.90 (m, 1H), 1.02 (dt, 1H), 1.11 (m, 2H), 1.13 (m, 1H), 1.16 (m, 1H), 1.18 (s, 1H), 1.22 (m, 1H), 1.25 (dt, 1H), 1.26 (m, 1H), 1.31 (m, 1H), 1.33 (m, 1H), 1.36 (dt, 1H), 1.38 (m, 1H), 1.46 (m, 1H), 1.49 (m, 1H), 1.56 (m, 1H), 1.59 (m, 1H), 1.61 (m, 1H), 1.62 (m, 1H), 2.01 (m, 1H), 2.05 (dt, 1H), 2.70 (s, 1H), 3.01 (s, 2H), 3.21 (s, 3H), 5.17 (d, 1H), 5.31 (d, 1H), 7.57 (d, 1H), 7.63 (s, 1H), 8.98 (s, 1H). ¹³C NMR (100 MHz, DMSO-d₆): δ 11.53, 13.59, 20.91, 22.99, 24.41, 28.56, 29.95, 32.14, 33.42, 35.50, 35.79, 37.13, 38.10, 40.04, 44.72, 54.01, 56.50, 58.19, 58.24, 60.02, 70.23, 82.22, 119.78, 123.77, 136.98, 203.06. MS (m/e) 428.6 (M, base peak).

### EXAMPLE 6

### 3α-Hydroxy-21-(1′-imidazolyl)-3β-methoxymethyl-5α-pregnan-20-one (6)

3α-Hydroxy-21-(1′-imidazolyl)-3β-methoxymethyl-5α-pregnan-20-one (6) was prepared according to Scheme 3 below:

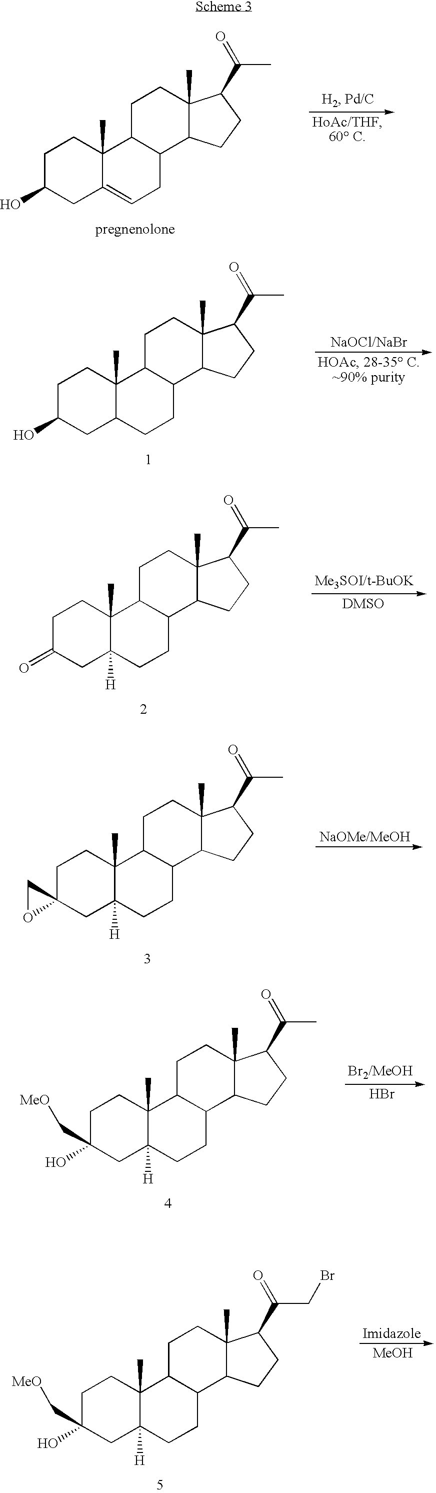

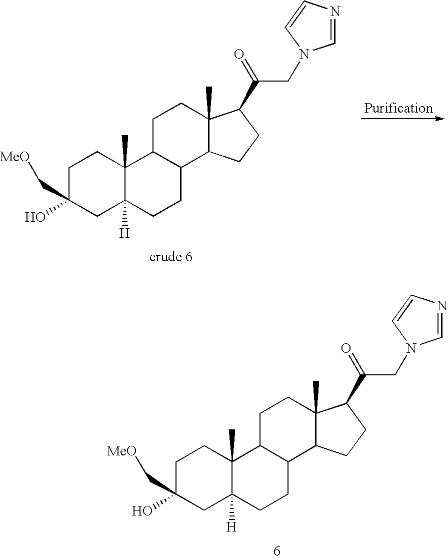

### (a) 3β-Hydroxy-5α-pregnan-20-one (1)

Pregnenolone (Changzhou Medical Raw Material Factory, Zhenglu Town, Changzhou City, Jiangsu, China) (26.5 kg) (HPLC, RT=7.0 min) was hydrogenated at approximately 60 psi in the presence of palladium on carbon (2.7 kg) in a mixture of tetrahydrofuran (255.1 kg) and glacial acetic acid (20.5 kg) at approximately 60° C. After completion of the reaction, the catalyst was removed by filtration and the filtrate was concentrated under reduced pressure. The residue (92.0 kg) containing 30-hydroxy-5α-pregnan-20-one (1) (HPLC, RT=8.0 min) and small amount of tetrahydrofuran/glacial acetic acid was used directly in the next reaction without further drying.

### (b) 5α-Pregnan-3,20-dione (2)

The wet product from the previous step (a) was dissolved in a mixture of tetrahydrofuran and glacial acetic acid (452.0 kg). At room temperature, the resulting solution was slowly treated with a mixture of sodium bromide (0.8 kg) and sodium hypochlorite (111.7 kg) while the reaction temperature was maintained below 40° C. After the reaction was completed, the excess sodium hypochlorite was quenched with sodium sulfite solution (112.0 kg of 11% in water), and the product, 5α-pregnan-3,20-dione (2) (HPLC, RT=9.3 min), precipitated. After filtration, the cake was washed with sodium hydroxide (5.6 kg in 116.8 kg of water), then with water (50.0 kg), and finally with heptanes (40.0 kg), and dried. The product (2) (20.0 kg) was obtained as an off-white solid with purity of approximately 90% (area percent, determined by GC).

### (c) 5(3R)-Spiro[oxirane-2′,5α-pregnan]-20-one (3)

Under nitrogen, potassium tert-butoxide (12.2 kg) was charged to a solution of trimethylsulfoxonium iodide (22.6 kg) in dimethyl sulfoxide (140.0 kg). The resulting mixture was heated to approximately 65° C. for approximately two hours and then cooled to room temperature. The product (2) from step (b) (20.0 kg) was charged to the above mixture while the internal reaction temperature was maintained between 25 and 35° C. After about two hours, water (350.0 kg) was added to the mixture and the product, 5(3R)-spiro[oxirane-2′,5α-pregnan]-20-one (3) (HPLC, RT=13.88 min), precipitated. The suspension was stirred at approximately 0° C. for approximately two hours, and filtered. The resulting precipitate was first washed with a mixture of water/methanol (4:1; v/v) (96 kg) and then with methanol (16 kg). Optional recrystallization from a mixture of methanol/acetone (5:1; v/v) (94 kg) and filtration gave the product (3) as an off-white wet cake (30.1 kg). The wet cake was washed with cold methanol (240.0 kg), then used in the next step without further purification.

### (d) 3α-Hydroxy-3β-methoxymethyl-5α-pregnan-20-one (4)

Under nitrogen, sodium methoxide (12.1 kg) was charged to a solution of 5(3R)-spiro[oxirane-2′,5α-pregnan]-20-one (3) (14.8 kg) in methanol (100.8 kg). The resulting mixture was heated at reflux for approximately three hours and cooled to room temperature. The reaction mixture was quenched with glacial acetic acid (25.9 kg), and the crude product (4) (HPLC, RT=11.19 min) precipitated from water (272.0 kg). The suspension was stirred at approximately 0° C. for approximately one hour, filtered and washed with water (127.0×2 kg). The crude product was dried and recrystallized from a mixture of ethyl acetate (9.4 kg) and heptanes (27.4 kg). 3α-Hydroxy-30-methoxymethyl-5α-pregnan-20-one (4) was washed with cold heptanes (3.0 kg), dried, and an off-white solid was obtained with purity of around 98% (area percent) (14.2 kg).

### (e) 21-Bromo-3α-hydroxy-3β-methoxymethyl-5α-pregnan-20-one (5)

A catalytic amount (0.9 kg) of aqueous hydrobromic acid solution (48%) was charged to a suspension of 3α-hydroxy-3β-methoxymethyl-5α-pregnan-20-one (4) (8.0 kg) in methanol (57.8 kg). The reaction mixture was shielded from light, and a solution of bromine (3.9 kg) in methanol (57.6 kg) was slowly added to the mixture over approximately two hours at room temperature. At the end of bromination, the light yellow solution was used directly in the next reaction without further purification.

### (f) 3α-Hydroxy-21-(1′-imidazolyl)-3β-methoxymethyl-5α-pregnan-20-one (6)

Imidazole (7.6 kg) was charged to a solution of 21-bromo-3α-hydroxy-3β-methoxymethyl-5α-pregnan-20-one (5) (HPLC, RT=14.7 min) in methanol prepared as described in the previous step. The resulting mixture was heated to approximately 55° C. under nitrogen. After completion of the reaction, the reaction mixture was concentrated under vacuum and cooled to room temperature. The residue was diluted with dichloromethane (100.1 kg), and the solution was extracted with methanol (3.7 kg) in water (30.0 kg). The organic layer was separated and co-distilled with ethyl acetate (7.0 kg) and n-heptane (15.2 kg) to remove dichloromethane. The resulting suspension diluted with a mixture of ethyl acetate/n-heptane (1:3 v/v) (40.0 kg) was cooled to approximately 0° C., stirred for approximately two hours and the solid precipitate filtered. The resulting cake was washed with another mixture of ethyl acetate/n-heptane (1:6; v/v) (7.9 kg) and dried.

The raw product (6) (7.0 kg) (HPLC, RT=3.7 min) was obtained as a pale yellow solid. A suspension of the raw product (6) in toluene (60.1 kg) was heated to reflux until a solution was achieved. Aluminum sulfate (0.6 kg) was added to the solution, and the resulting suspension was stirred and filtered when the mixture was at approximately 100° C. The filtrate was concentrated under reduced pressure to a suspension (about 40.0 L) and isopropyl ether (72.6 kg) was added. After cooling at approximately 0° C. for about three hours, the solid was collected by filtration; the cake was washed with isopropyl ether (9.6 kg) and dried. The crude product (6) (6.4 kg) was obtained as a pinkish solid with purity around 85% (area percent as determined by HPLC).

The crude product (6) (4.8 kg) was further dissolved in a mixture of methanol (1.0 kg) in dichloromethane (42.6 kg). The resulting solution was eluted through a Biotage flash column with a mixture of methanol (22.4 kg) in dichloromethane (707.0 kg). The fractions containing pure product (6) were collected and concentrated under reduced pressure. The residue was suspended with isopropyl ether (3.0 kg) and the formed solid was collected by filtration, washed with cold isopropyl ether (26.0 kg) and dried. The solid was re-dissolved in methanol (37.7 kg), and treated with activated carbon (0.4 kg). Spent activated carbon was filtered, washed with methanol (6.0 kg), and the filtrate concentrated. Isopropyl ether (26.6 kg) was added to the concentrate to initiate recrystallization. After collection by filtration, the cake was washed with cold isopropyl ether (3.0 kg) and dried to give pure 3α-hydroxy-21-(1′-imidazolyl)-3β-methoxymethyl-5α-pregnan-20-one (6) obtained as a crystalline solid (3.1 kg) of purity around 99% (w/w).

### EXAMPLE 7

### 17α-Acetyl-3α-hydroxy-21-(1′-imidazolyl)-3β-methoxymethyl-5α-pregnan-20-one (7)

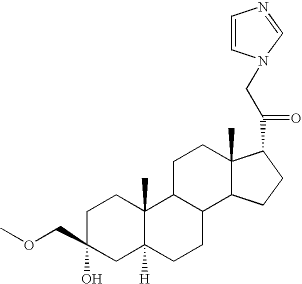

### (a) 17α-Acetyl-3α-hydroxy-3β-methoxymethyl-5α-pregnan-20-one (8)

17α-Acetyl-3α-hydroxy-3β-methoxymethyl-5α-pregnan-20-one (8) was purified from the evaporated re-crystallization filtrate of Example 4 (4.57 g, containing 64% of 17α isomer) by column chromatography (SiO₂, 210 g; eluted with 5% acetone in dichloromethane) to obtain pure 17α-acetyl-3α-hydroxy-3β-methoxymethyl-5α-pregnan-20-one (2.8 g). ¹H NMR (400 MHz, CDCl₃): δ 0.74 (s, 3H), 0.76 (m, 1H), 0.91 (s, 3H), 1.04 (m, 1H), 1.14-1.32 (m, 11H), 1.40-1.60 (m, 5H), 1.65-1.80 (m, 4H), 1.85 (m, 1H), 1.98 (s, 1H), 2.12 (s, 3H), 2.77 (d, J=8 Hz, 1H), 3.17 (s, 2H), 3.38 (s, 3H). ¹³C NMR (75 MHz, CDCl₃): δ 11.5, 20.92, 20.95, 24.27, 25.89, 28.49, 30.20, 32.20, 32.70, 33.30, 35.39, 35.77, 35.99, 37.13, 40.07, 45.79, 50.35, 53.41, 59.41, 61.44, 70.97, 82.00, 212.59.

### (b) 17α-Acetyl-21-bromo-3α-hydroxy-3β-methoxymethyl-5α-pregnan-20-one (9)

17α-Acetyl-21-bromo-3α-hydroxy-3β-methoxymethyl-5α-pregnan-20-one (9) was prepared as follows. Methanol (50 mL) and 17α-acetyl-3α-hydroxy-3β-methoxymethyl-5α-pregnan-20-one (8) (4.9 g) were charged to a 100 mL flask. The resulting suspension was stirred at 20-30° C. Bromine (2.5 g) was slowly added as a solution in methanol (40 mL) while the internal temperature of the reaction mixture was controlled at 20-30° C. After the bromination was completed (monitored by TLC), the reaction mixture was directly used in the next step without any purification.

### (c) 17α-Acetyl-3α-hydroxy-21-(1′-imidazolyl)-3β-methoxymethyl-5α-pregnan-20-one (7)

The reaction mixture from step (b) was charged with imidazole (5.08 g) and the resulting suspension was stirred at 55-65° C. (internal temperature) overnight. After the reaction was completed, the reaction mixture was concentrated under reduced pressure to obtain an oil, which was re-dissolved in dichloromethane (250 mL) and extracted with water (250 mL). The organic layer was separated, concentrated under reduced pressure to the crude product, which was purified by column chromatography (SiO₂, 210 g; eluted first with 5% methanol/95% CH₂Cl₂ and then with 10% methanol/90% CH₂Cl₂) to afford an oil. Upon the addition of heptane, a suspension was formed, which was filtered and dried to yield the product (7) (3.4 g) in 59% yield for two steps. ¹H NMR (400 MHz, CDCl₃): δ 0.75 (s, 3H), 0.76 (m, 1H), 0.94 (s, 3H), 1.04 (m, 1H), 1.15-1.36 (m, 9H), 1.48 (m, 4H), 1.67 (m, 2H), 1.79-1.91 (m, 6H), 2.75 (dd, J=8.0, 2.8 Hz, 1H), 3.18 (s, 2H), 3.39 (s, 3H), 4.62 (d, J=18.4 Hz, 1H), 4.78 (d, J=18.4 Hz, 1H), 6.87 (s, 1H), 7.11 (s, 1H), 7.44 (s, 1H).

### EXAMPLE 8

### 2,3-Dihydro-1,3-di(3α-hydroxy-3β-methoxymethyl-5α-pregnan-20-one-21-yl)-imidazonium bromide (10)

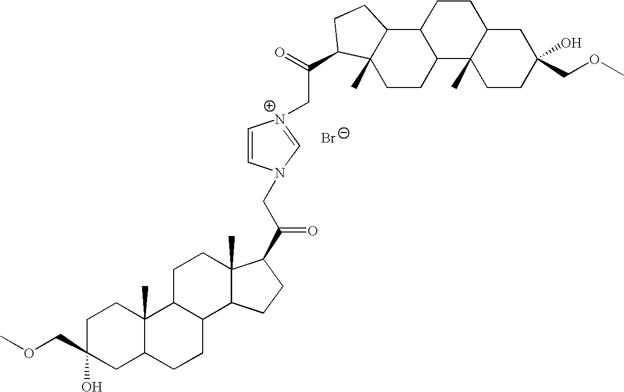

23.1 g of 21-bromo-3α-hydroxy-3β-methoxymethyl-5α-pregnan-20-one (5), 15 g of 3α-hydroxy-21-(1′-imidazolyl)-3β-methoxymethyl-5α-pregnan-20-one (6), and 225 mL of THF were charged to a suitable reactor. The suspension was stirred and heated to reflux (about 60° C.) overnight. The reaction was monitored by TLC (elution system: 3% acetone in dichloromethane). After the reaction was complete, the reaction mixture was concentrated to about 100 mL in volume under reduced pressure, and added with a 75 mL mixture of ethyl acetate/heptanes (v/v=3/7) for solvent swap. The suspension was concentrated to about 75 mL under reduced pressure, filtered and washed with 30 mL of ethyl acetate/heptanes co-solvent (v/v=1/9). The wet cake was dried in a vacuum oven at below 60° C. for 48 hours. 30.04 g of the desired product was gained as an off-white solid with a 97.85% yield. ¹H NMR (400 MHz, MeOH-d₄): δ 0.66 (s, 6H, 2×CH₃), 0.73 (s, 6H, 2×CH₃), 1.08-0.80 (m, 4H), 1.90-1.10 (m, 36H), 2.20-2.00 (m, 4H), 2.69 (t, J=8.8 Hz, 2H, 17C—H), 3.16 (s, 4H, 2×OCH₂), 3.36 (s, 6H, 2×OCH₃), 5.20 (d, J=18.4 Hz, 2H), 5.29 (d, J=18.4 Hz, 2H), 7.32 (s, 1H), 9.30 (s, 1H).

### EXAMPLE 9

### 3α-Hydroxy-21-(1′-imidazolyl)-3β-methylthioethyl-5α-pregnan-20-one (11)

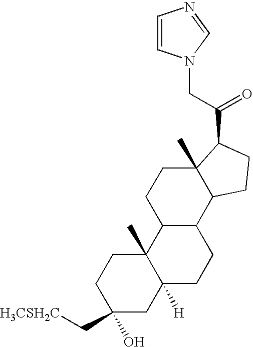

3α-Hydroxy-21-(1′-imidazolyl)-3β-methylthioethyl-5α-pregnan-20-one (11) was isolated from the raw reaction product 3α-hydroxy-21-(1′-imidazolyl)-3β-methoxymethyl-5α-pregnan-20-one (6) prepared in Example 6, step (f), by HPLC using Xterra columns 18, 100×30 mm. The conditions were the following: detector: 220 nm; flow rate: 10 mL/min, mobile phase: 41% ACN/H₂O with 0.1% TFA; injection: 40 to 100 mg in DMSO. Compound (11) eluted at 17 to 18 minutes. The fraction was collected and evaporated under reduced pressure. FTMS: 459.3040 m/z. ¹H NMR (400 MHz, MeOH-d₄): δ 0.46 (s, 3H), 0.60 (s, 3H), 0.70 (m, 1H), 0.86 (m, 1H), 1.05-1.36 (m, 17H), 1.53 (m, 2H), 1.60-2.20 (m, 7H), 2.42 (m, 2H), 2.60 (s, 1H), 4.72 (d, 1H), 4.82 (d, 1H), 6.84 (m, 2H), 7.38 (s, 1H).

### EXAMPLE 10

### 3α-Hydroxy-21-(1′-imidazolyl)-3β-hydroxymethyl-5α-pregnan-20-one (12)

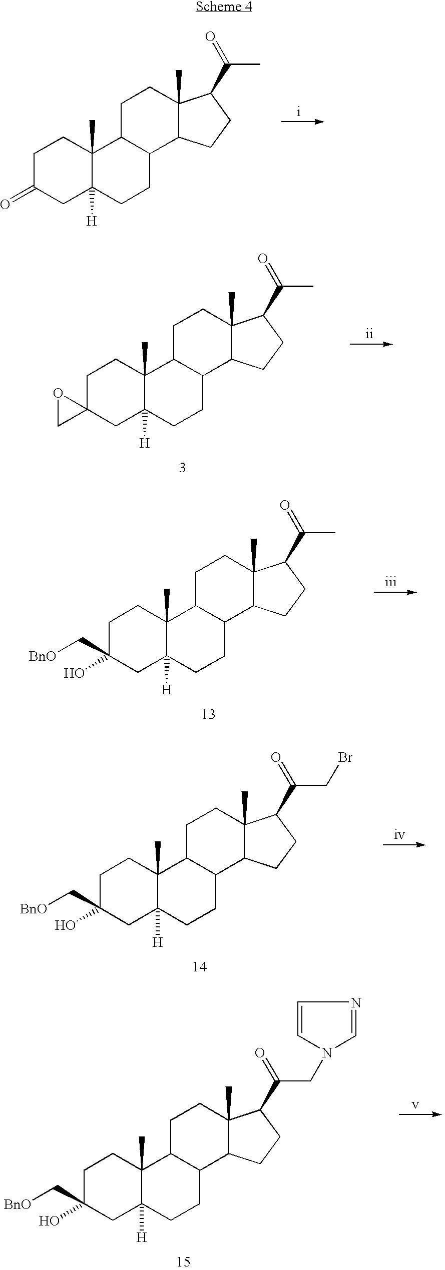

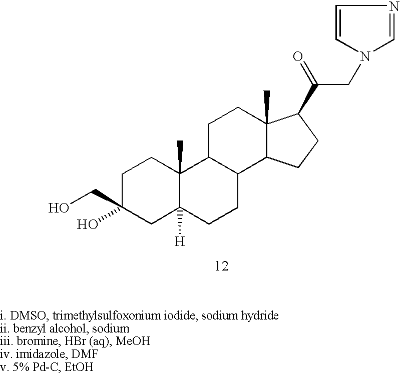

### (a) 5(3R)-Spiro[oxirane-2′,5α-pregnan]-20-one (3)

A solution of 51.0 g of trimethylsulfoxonium iodide and 7.88 g of 60% NaH (dispersion in mineral oil) in 450 mL of DMSO was stirred at room temperature for 1.5 hours. A suspension of 15.0 g of 5α-pregnan-3,20-dione in 150 mL of DMSO was added dropwise to the solution, and the solution was stirred for an additional 4 hours at room temperature. The solution was then poured into ice water, and the mixture was extracted with ether. The combined extracts were washed with brine, dried (Na₂SO₄) and concentrated. Recrystallization of the residue from 1:1 (v:v) methanol:acetone yielded 9.85 g of the title compound (3) as white crystals, m.p. 161-163° C.

### (b) 3β-(Benzyloxymethyl)-3α-hydroxy-5α-pregnan-20-one (13)

A solution of 9.12 g of 5(3R)-spiro[oxirane-2′,5α-pregnan]-20-one (3) in 450 mL of benzyl alcohol, in which 1.20 g of sodium had been dissolved, was heated at 80° C. for 16 hours. After cooling to room temperature, 5 mL of acetic acid was added. The solvent was removed by distillation under reduced pressure, then methylene chloride and water were added. The mixture was extracted with methylene chloride, the combined extracts were washed with saturated NaHCO₃, dried (Na₂SO₄), and concentrated. Recrystallization of the residue from 1:1 hexane:acetone (v:v) yielded 5.88 g of the title compound (13) as white crystals, m.p. 124-126° C.

### (c) 21-Bromo-3β-(benzyloxymethyl)-3α-hydroxy-5α-pregnan-20-one (14)

A solution of 0.58 mL of 48% HBr in 1.3 mL of methanol was added to a suspension of 4.0 g of compound (13) in 80 mL of methanol followed by a solution of 1.58 g of bromine in 13 mL of methanol. The mixture was stirred at room temperature for 1.5 hours and then a solution of 8.3 g of sodium acetate in 20 mL of water was added to the mixture, followed by 100 mL of water. The mixture was extracted with ether, the combined extracts were dried (Na₂SO₄), and concentrated, yielding 4.58 g of the title compound (14) as a white solid.

### (d) 21-(1′-Imidazolyl)-3β-(benzyloxymethyl)-3α-hydroxy-5α-pregnan-20-one (15)

A solution of 3.0 g of compound (14) in 25 mL of DMF was added to a solution of 12.0 g of imidazole in 25 mL of DMF at 0° C. The solution was allowed to warm to room temperature overnight and it was then poured into 100 mL of water containing 0.25 g of NaOH. The mixture was extracted with ethyl acetate, the combined extracts were washed with water, dried (Na₂SO₄), and concentrated. Purification of the residue by column chromatography (basic alumina, 5% methanol in ethyl acetate) yielded 1.71 g of the title compound (15) as a white solid.

### (e) 21-(1′-Imidazolyl)-3β-(hydroxymethyl)-3α-hydroxy-5α-pregnan-20-one (12)

A solution of 1.30 g of compound (15) in ethanol was hydrogenated at 35 psi over 0.71 g of 10% palladium on activated carbon for 3 days. The mixture was then filtered, the filtrate concentrated and the residue purified by column chromatography (neutral alumina, 5% methanol in ethyl acetate), yielding 0.81 g of the title compound (12) as a white solid. Recrystallization of this solid from acetone gave 187 mg of the desired product (12). LCMS (positive ion): m/z 415 W).

¹H-NMR (300 MHz, DMSO-d₆): δ 7.53-7.46 (s, 1H), 7.05-6.98 (s, 1H), 6.90-6.81 (s, 1H), 5.08-4.97 (d, J=18.5 Hz, 1H), 4.93-4.82 (d, J=18.5 Hz, 1H), 4.48-4.41 (m, 1H), 3.79-3.74 (s, 1H), 3.12-3.04 (m, 2H), 2.72-2.63 (m, 1H), 2.11-1.99 (m, 2H), 1.72-1.10 (bm), 1.02-0.87 (m, 2H), 0.79-0.72 (m, 1H), 0.72-0.69 (s, 3H), 0.60-0.54 (s, 3H).

### EXAMPLE 11

### 21-(1′-Imidazolyl)-3β-methoxymethyl-5α-pregnan-3α,20-diol (16)

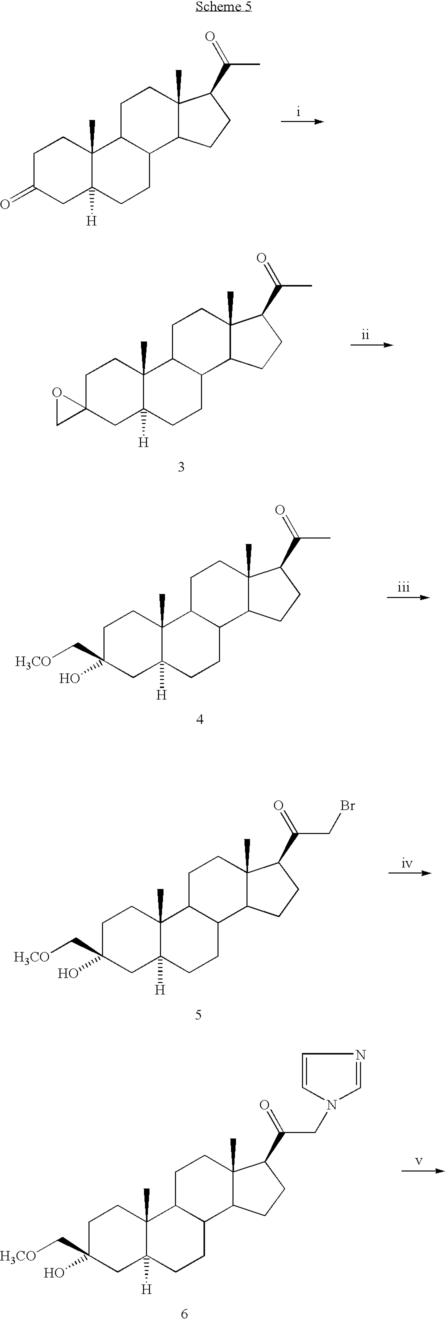

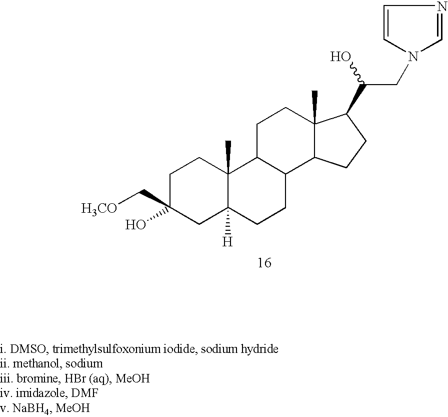

### (a) 5(3R)-Spiro[oxirane-2′,5α-pregnan]-20-one (3)

A solution of 16.8 g of trimethylsulfoxonium iodide and 2.82 g of 60% NaH (dispersion in mineral oil) in 200 mL of DMSO was stirred at room temperature for 1.5 hours. A suspension of 4.98 g of 5α-pregnan-3,20-dione in 100 mL of DMSO was then added dropwise to the solution and the solution was stirred for an additional 4 hours at room temperature. The solution was then poured into ice water, the mixture was extracted with ether, the combined extracts were washed with brine, dried (Na₂SO₄) and concentrated. Recrystallization of the residue from 1:1 methanol:acetone yielded 3.48 g of the title compound (3) as white crystals, m.p. 161-163° C.

### (b) 3α-Hydroxy-3β-(methoxymethyl)-5α-pregnan-20-one (4)

A solution of 3.48 g of compound (3) in 400 mL of methanol, in which 0.60 g of sodium had been dissolved, was heated at 80° C. for 16 hours. After cooling to room temperature, 5 mL of acetic acid was added. The solvent was removed by rotoevaporation, and methylene chloride and water were then added. The mixture was extracted with methylene chloride, the combined extracts were washed with saturated NaHCO₃, dried (Na₂SO₄) and concentrated. Recrystallization of the residue from 1:1 hexane:acetone yielded 2.19 g of the title compound (4) as white crystals, m.p. 163-164° C.

### (c) 21-Bromo-3α-hydroxy-3β-(methoxymethyl)-5α-pregnan-20-one (5)

A solution of 0.4 mL of 48% HBr in 0.75 mL of methanol was added to a solution of 2.0 g of compound (4) in 20 mL of methanol, followed by dropwise addition over 1.5 hours of a solution of 1.00 g of bromine in 13 mL of methanol. The mixture was stirred at room temperature for an additional 0.5 hour, and a solution of 3.0 g of sodium acetate in 10 mL of water was then added, followed by 50 mL of water. The mixture was extracted with ether, the combined extracts were dried (Na₂SO₄) and concentrated, yielding 1.8 g of the title compound (5) as a white solid.

### (d) 3α-Hydroxy-21-(1′-imidazolyl)-3β-(methoxymethyl)-5α-pregnan-20-one (6)

A solution of compound (5) in 10 mL of DMF as added to a solution of 2.15 g of imidazole in 10 mL of DMF at 0° C. The solution was allowed to warm to room temperature overnight and then poured into 50 mL of water containing 0.2 g of NaOH. The mixture was extracted with ethyl acetate, the combined extracts were washed with water, dried (Na₂SO₄) and concentrated. Purification of the residue by column chromatography (basic alumina, 3% methanol in methylene chloride) yielded 1.09 g of the title compound (6) as a white solid.

### (e) 21-(1′-Imidazolyl)-3β-(methoxymethyl)-5α-pregnan-3α,20-diol (16)

A solution of 0.65 g of sodium borohydride in 20 mL of methanol was added dropwise to a solution of 1.08 g of compound (6) in 100 mL of methanol. The solution was stirred for 2 hours at room temperature, and then the solvent was removed by rotoevaporation. Methylene chloride and water were added, the layers were separated, and the aqueous layer was extracted with methylene chloride. The combined extracts were dried (Na₂SO₄), concentrated and the residue was purified by column chromatography (basic alumina, 3% methanol in methylene chloride). The fractions containing the desired material were combined, concentrated and the residue was recrystallized from 1:1 methylene chloride:acetone (v:v), yielding 0.75 g of the title compound (16) as white crystals. LCMS (positive ion): m/z 431 (MH⁺). ¹H-NMR (300 MHz, CDCl₃): δ 7.52-7.45 (s, 1H), 7.07-7.00 (s, 1H), 6.98-6.92 (s, 1H), 4.07-3.96 (m, 1H), 3.80-3.70 (m, 2H), 3.42-3.34 (s, 3H), 3.20-3.13 (s, 2H), 2.12-1.90 (m, 3H), 1.81-0.88 (bm), 0.86-0.58 (m, 7H).

### EXAMPLE 12

### 3β-(ethoxymethyl)-3α-hydroxy-5α-pregnan-20-one (4)

Synthesis 1: 5 g of 5(3R)-spiro[oxirane-2′,5α-pregnan]-20-one (3) (1.0 eq.) was reacted with sodium methoxide (2.5 eq.) in 15 mL of MeOH at reflux temperature (about 68° C.) for 2 hours. The 17β/17α ratio of the product was 79.24/20.33.

Synthesis 2: 2.5 g of 5(3R)-spiro[oxirane-2′,5α-pregnan]-20-one (3) (1.0 eq.) was reacted with sodium methoxide (2.5 eq.) in 15 mL of MeOH/THF (v/v 4/1) at reflux temperature (about 60-65° C.) for 2 hours. The 17β/17α ratio of the product was 78.68/20.51.

Synthesis 3: 1.0 g of 5(3R)-spiro[oxirane-2′,5α-pregnan]-20-one (3) (1.0 eq.) was reacted with sodium methoxide (2.5 eq.) in 15 mL of MeOH/THF (v/v 4/1) at 35-40° C. for 8 hours. The 17β/17α ratio of the product was 88.37/11.10.

Synthesis 4: 1.0 g of 5(3R)-spiro[oxirane-2′,5α-pregnan]-20-one (3) (1.0 eq.) was reacted with a combination of sodium methoxide (2.5 eq.) and Mg(OMe)₂ (0.5 eq.) in 15 mL of MeOH at 35-40° C. for 7-8 hours. The 17β/17α ratio of the product was 87.99/11.86.

Synthesis 5: 1.0 g of 5(3R)-spiro[oxirane-2′,5α-pregnan]-20-one (3) (1.0 eq.) was reacted with a combination of sodium methoxide (2.5 eq.) and ZnCl₂ (0.5 eq.) in 15 mL of MeOH at 35-40° C. for 7-8 hours. The 17β/17α ratio of the product was 90.76/8.39.

Synthesis 6: 1.0 g of 5(3R)-spiro[oxirane-2′,5α-pregnan]-20-one (3) (1.0 eq.) was reacted with lithium hydroxide (2.5 eq.) in 15 mL of MeOH at reflux temperature (about 60-65° C.) for 4 hours. The 17β/17α ratio of the product was 79.91/19.21.

Synthesis 7: 1.0 g of 5(3R)-spiro[oxirane-2′,5α-pregnan]-20-one (3) (1.0 eq.) was reacted with sodium hydroxide (2.5 eq.) in 15 mL of MeOH at reflux temperature (about 60-65° C.) for 3 hours. The 17β/17α ratio of the product was 79.28/19.87.

Synthesis 8: 1.0 g of 5(3R)-spiro[oxirane-2′,5α-pregnan]-20-one (3) (1.0 eq.) was reacted with sodium hydroxide (2.5 eq.) in 15 mL of MeOH at about 35-40° C. for 8 hours. The 17β/17α ratio of the product was 93.68/4.92.

Synthesis 9: 100.0 g of 5(3R)-spiro[oxirane-2′,5α-pregnan]-20-one (3) (1.0 eq.) was reacted with sodium hydroxide (2.5 eq.) in 1.0 L of MeOH at about 35-45° C. for 12-15 hours. Two similar reactions were conducted. The 17β/17α ratios of the product were 87.6/8.1 and 85.7/10.4.

### EXAMPLE 13

### Epimerization of Isomeric Mixture of 3α-hydroxy-3β-methoxymethyl-5α-pregnan-20-one

The mother liquor obtained after the filtration in Example 6 step (d) was evaporated to dryness (the ratio of 17β/17α=about 35.6/63.5) and the recovered solid (100.0 g) was suspended in 900 mL of methanol. 38.2 g (1.0 eq.) of potassium carbonate was added into the suspension and the mixture was heated to reflux (about 60-70° C.) for 5 hours. After refluxing 5 hours, the ratio of 17β/17α had changed to about 80/20. The reaction solution was cooled to room temperature and 900 mL of water was slowly added to precipitate the product. The product was filtered and dried in a vacuum oven until the water content was less than 2%. Yield 93.8 g (about 94%) having the 17β/17α ratio of 79/20.

9.3 g of the epimerized mixture was further re-crystallized from ethyl acetate/heptanes (1/1) solution. The reaction mixture was heated to reflux (70-80° C.) and then cooled to room temperature for 2-3 hours. The solution was further cooled to 0-5° C. for an hour. The product was filtered, washed with heptanes, and dried in a vacuum oven. 17β was obtained in about 80% yield and its purity was more than 98% (area percent).

### EXAMPLE 14

### Synthesis of 3α-hydroxy-21-(1′-imidazolyl)-3β-(methoxymethyl)-5α-pregnan-20-one (6) Using Li-imidazole

Imidazole (4.0 eq.) was dissolved in THF and transferred to a mixture of LiH (4.05 eq.) in THF, and the resulting mixture was heated to reflux for about 1-2 hours under nitrogen gas. Hydrogen was generated during the procedure. After 1-2 hours, the resulting solution was cooled to 0-10° C. and 21-bromo-3α-hydroxy-3β-(methoxymethyl)-5α-pregnan-20-one (5) (1.0 eq.) in THF solution was quickly added to the mixture. After the reaction was complete (monitored by TLC), the reaction was quenched by water and NH₄Cl. The organic layer was washed twice with 5% aqueous NaCl solution. The organic layer was concentrated and toluene was added to precipitate the title product 3α-hydroxy-21-(1′-imidazolyl)-3-(methoxymethyl)-5α-pregnan-20-one (6). After filtration, the wet cake was dried in a vacuum oven, and the purity was determined by HPLC.

### EXAMPLE 15

### Synthesis of 3α-hydroxy-21-(1′-imidazolyl)-3β-(methoxymethyl)-5α-pregnan-20-one (6) Using Li-imidazole

The scale-up at 150 g of 21-bromo-3α-hydroxy-3β-(methoxymethyl)-5α-pregnan-20-one (5) was performed in two batches using the procedure of Example 14 at −10° C. The addition time of a solution of compound (5) in THF was controlled at about 35 minutes and the reaction mixture was maintained at the same temperature for about 30 minutes. The reaction mixture was monitored and sampled every 10 minutes. When the reaction was complete, the mixture was quenched by NH₄Cl/NaCl/H₂O solution. The quench was performed after about 30 minutes.

The results show that the reaction was complete after about 5-10 minutes and the impurities would increase with longer reaction time.

The crude product (6) was precipitated by adding toluene and the purity increased from about 93% to about 95%. When the crude product (6) was crystallized from MeOH/isopropylether (4/12, v/v, based on product) or MeOH/acetone/isopropylether (1/3/10, v/v) solvent system, and then slurried in acetone/H₂O (1/3, v/v), pure product was obtained as a light yellow powder with >99.5% purity (area percent) and with about 98% purity by assay.

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

1. A process comprising:
reacting a compound of Formula II: 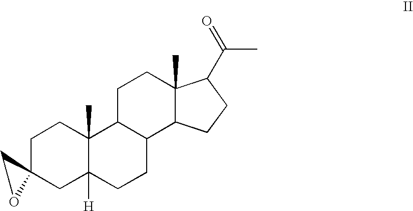
with a reagent comprising one or more alkali metal alkoxide, alkaline-earth metal alkoxide or alkali metal hydroxide, and optionally a Lewis acid, in an appropriate solvent to open the oxirane ring without affecting the 20-position keto group, to provide a reaction mixture comprising a compound of Formula III: 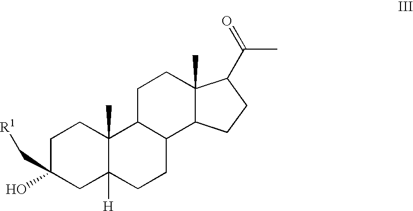
wherein R¹ is an alkoxy group, and optionally isolating the desired 17β or 17α isomer.

2. The process of claim 1, wherein the reagent comprises an alkali metal alkoxide.

3-4. (canceled)

5. The process of claim 1, wherein the reagent comprises an alkali metal hydroxide.

6. The process of claim 1, wherein the solvent is methanol or a mixture of methanol with an aprotic, polar solvent.

7. (canceled)

8. The process of claim 2, wherein the process is conducted at reflux temperature.

9. The process of claim 8, wherein the reaction time is from about 3 hours to about 8 hours.

10. The process of claim 5, wherein the process is conducted at about 35 to about 45° C.

11. The process of claim 10, wherein the reaction time is from about 8 hours to about 15 hours.

12. The process of claim 1, wherein the compound of Formula II is primarily the 5α isomer.

13. The process of claim 12, wherein the compound is primarily the 17β isomer.

14. The process of claim 1, further comprising isolating the 17β isomer of the compound of Formula III from the reaction mixture to obtain a crude product, and optionally re-crystallizing the 17β isomer from the crude product.

15. The process of claim 1, wherein the purity of the compound of Formula II is at least 85% (area percent) as determined by HPLC.

16. (canceled)

17. The process of claim 1, wherein the compound of Formula II is reacted with NaOH in methanol.

18. The process of claim 17, wherein the reaction temperature is about 35 to about 45° C.

19. The process of claim 14, wherein the 17α isomer is epimerized and recycled.

20. A process comprising:
reacting a compound of Formula III: 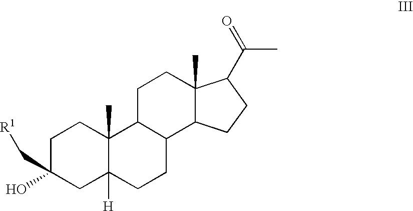
wherein R¹ is an alkoxy group, with a bihalogen in the presence of a haloacid to form a product mixture comprising a halogenated derivative having the Formula IV: 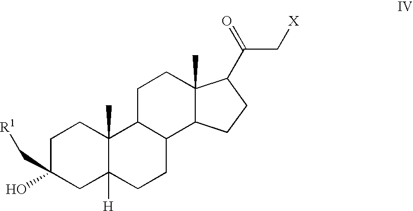
wherein R¹ is as defined above and X is a halogen, and optionally isolating and purifying the compound of Formula IV.

21. The process of claim 20, wherein the compound of Formula IV is the 5α and 17β isomer.

22. The process of claim 20, wherein the bihalogen is Br₂ in a methanol solution and the haloacid is HBr.

23. The process of claim 20, wherein the compound of Formula IV is isolated by adding water to the mixture to obtain a precipitate.

24. The process of claim 23, further comprising filtering the precipitate and washing the precipitate with a solvent selected from the group consisting of water, methanol, acetone, THF, isopropyl ether, and n-heptane and mixtures thereof.

25. (canceled)

26. A process for making 3α-hydroxy-3β-alkoxymethyl-21-substituted-5α-pregnan-20-one compound or 3α-hydroxy-3β-alkoxymethyl-21-substituted-5β-pregnan-20-one compound of Formula I: 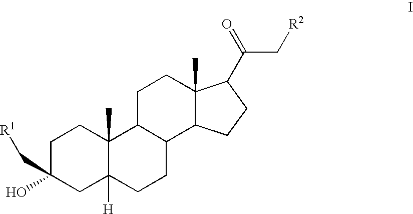
wherein:
R¹ is an alkoxy group; and R² is an optionally substituted, N-attached heteroaryl, comprising
reacting a product mixture comprising a compound of Formula IV or reacting an isolated compound of Formula IV: 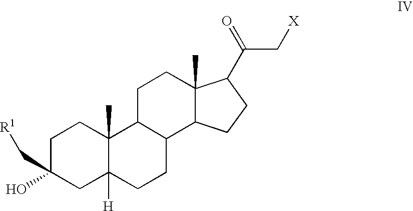
wherein R¹ is an alkoxy group and X is a halogen, with a nitrogen-containing, optionally substituted heteroaryl compound or an alkali metal salt of a nitrogen-containing, optionally substituted heteroaryl compound to obtain a mixture comprising a compound of Formula I, and optionally isolating and purifying the compound of Formula I.

27. The process of claim 26, wherein the isolated compound of Formula IV is the reactant.

28. The process of claim 27, wherein the compound is 5α and 17β isomer.

29. The process of claim 26, wherein the nitrogen-containing heteroaryl is selected from the group consisting of oxazole, thiazole, tetrazole, imidazole, pyrrole, pyridine, pyrimidine, quinoline and isoquinoline, each of which are optionally substituted.

30. (canceled)

31. The process of claim 26, wherein the nitrogen-containing, optionally substituted heteroaryl is in the form of an alkali metal salt.

32-33. (canceled)

34. The process of claim 31, wherein the compound of Formula I is isolated from the reaction mixture by precipitating with a suitable solvent and collecting the precipitate.

35. (canceled)

36. The process of claim 34, wherein the precipitate is purified by recrystallizing from a solvent selected from the group consisting of methanol, isopropylether, acetone and mixtures thereof to obtain a purified compound of Formula I.

37. (canceled)

38. The process of claim 1, wherein the compound of Formula II is prepared by reacting a compound of Formula V: 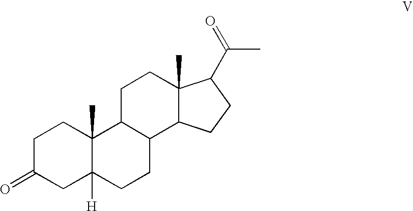
with an ylide.

39. The process of claim 38, wherein the ylide is prepared by mixing trimethylsulfoxonium iodide in an aprotic solvent or mixtures thereof with potassium tert-butoxide under a dry atmosphere to selectively form an oxirane ring at the 3-position keto group of the compound of Formula V to form a mixture comprising a compound of Formula II, and further purifying the compound of Formula II by recrystallizing from a polar or weakly polar solvent or mixtures thereof.

40-41. (canceled)

42. A process, comprising reacting 5(3R)-spiro[oxirane-2′,5α-pregnan]-20-one of formula: 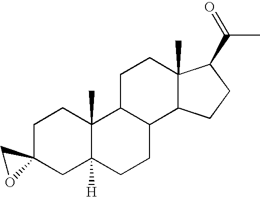
with NaOMe or NaOH in an appropriate solvent to obtain a mixture comprising 3αhydroxy-3β-methoxymethyl-5α-pregnan-20-one of the formula

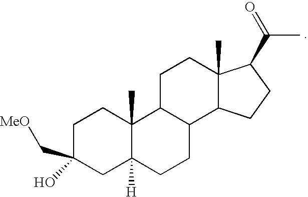

43. The process of claim 42, wherein the solvent is methanol.

44-45. (canceled)

46. The process of claim 42, further comprising purifying the 171 isomer of 3α-hydroxy-3β-methoxymethyl-5α-pregnan-20-one.

47. A process, comprising reacting 3α-hydroxy-3β-methoxymethyl-5α-pregnan-20-one of the formula 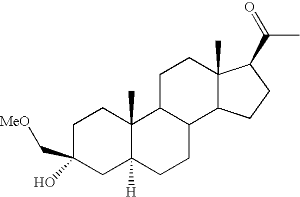
with Br₂ in the presence of HBr to obtain a mixture comprising 21-bromo-3α-hydroxy-3β-methoxymethyl-5α-pregnan-20-one of formula 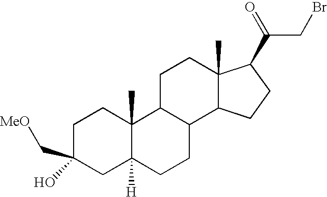
isolating the brominated compound from the mixture, and optionally purifying the brominated compound.

48. The process of claim 47, wherein the brominated compound is isolated by adding water to the mixture to obtain a precipitate and collecting the precipitate.

49. The process of claim 48, wherein the precipitate is washed with a solvent selected from the group consisting of water, methanol, acetone, THF, isopropyl ether, n-heptane and mixtures thereof.

50. A process for preparing 3α-hydroxy-21-(1′-imidazolyl)-3β-methoxymethyl-5α-pregnan-20-one of the following formula: 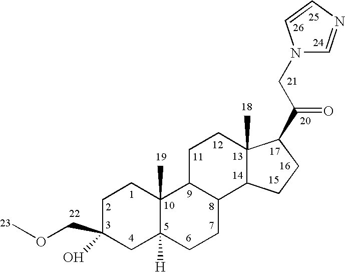
comprising reacting 21-bromo-3α-hydroxy-3β-methoxymethyl-5α-pregnan-20-one of formula 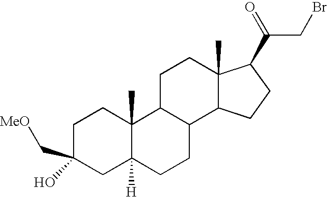
with a lithium salt of imidazole to obtain a mixture comprising the product 3α-hydroxy-21-(1′-imidazolyl)-3β-methoxymethyl-5α-pregnan-20-one.

51. The process of claim 50, further comprising quenching the reaction with aqueous NH₄Cl/NaCl solution for an appropriate time period.

52. The process of claim 51, further comprising purifying the product from the mixture by precipitating with a suitable solvent to obtain a crude product.

53. (canceled)

54. The process of claim 52, further comprising recrystallizing the crude product from a solvent selected from the group consisting of methanol, isopropylether, acetone, and mixtures thereof.

55. (canceled)

56. The process of claim 1, wherein the compound of Formula II is 5(3R)-spiro[oxirane-2′,5α-pregnan]-20-one.

57. The process of claim 20, wherein the compound of Formula III is 3α-hydroxy-3β-methoxymethyl-5α-pregnan-20-one.

58. The process of claim 26, wherein the compound of Formula IV is 21-bromo-3α-hydroxy-3β-methoxymethyl-5α-pregnan-20-one.

59. The process of claim 26, wherein the compound of Formula I is 3α-hydroxy-21-(1′-imidazolyl)-3β-methoxymethyl-5α-pregnan-20-one.

60. A compound of Formula VIII 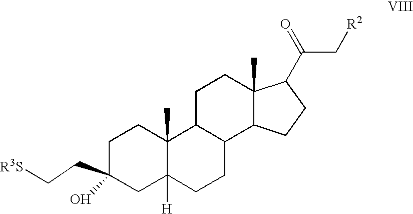
or a pharmaceutically acceptable salt, prodrug or solvate thereof, wherein R² is an optionally substituted, N-attached heteroaryl and R³ is an alkyl group.

61-63. (canceled)

64. The compound of claim 60, wherein the compound is 3α-hydroxy-21-(1′-imidazolyl)-3β-methylthioethyl-5α-pregnan-20-one having the formula 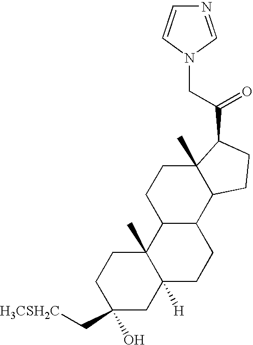
or a pharmaceutically acceptable salt, prodrug or solvate thereof.

65. A compound of Formula X 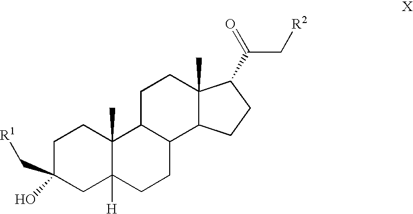
or a pharmaceutically acceptable salt, prodrug or solvate thereof, wherein R¹ is an alkoxy group and R² is an optionally substituted, N-attached heteroaryl.

66. The compound of claim 65, wherein the compound is 17α-acetyl-3α-hydroxy-21-(1′-imidazolyl)-3β-methoxymethyl-5α-pregnan-20-one or a pharmaceutically acceptable salt, prodrug or solvate thereof.

67. A compound having the Formula XI 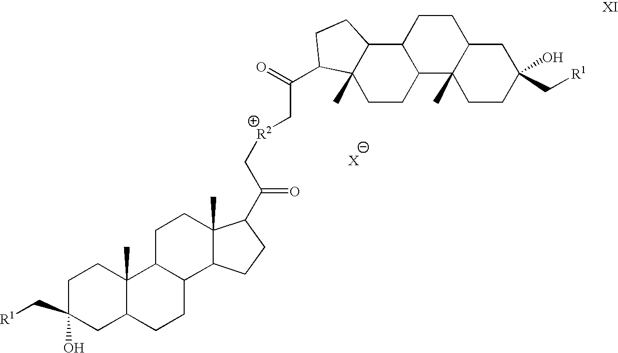
or a pharmaceutically acceptable salt, prodrug or solvate thereof, wherein each R¹ is independently an alkoxy group and R² is an optionally substituted, N-attached heteroaryl, provided that R² is a heteroaryl group having at least two nitrogen atoms wherein each of the two nitrogen atoms is substituted with one of the tails of Formula XI.

68. (canceled)

69. The compound of claim 67 having the formula 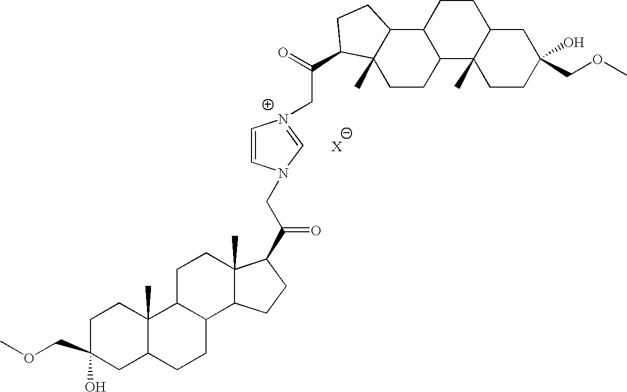
or a pharmaceutically acceptable salt, prodrug or solvate thereof, wherein X is a halogen.

70. (canceled)

71. A compound having the following Formula XII 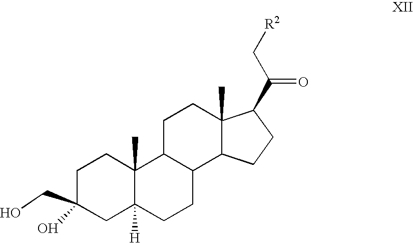
or a pharmaceutically acceptable salt or solvate thereof, wherein R² is an optionally substituted, N-attached heteroaryl.

72. A compound having the following Formula XIII 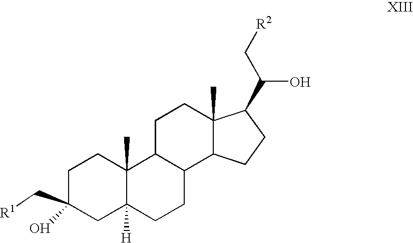
or a pharmaceutically acceptable salt or solvate thereof, wherein R¹ is an alkoxy group and R² is an optionally substituted, N-attached heteroaryl.

73-75. (canceled)

76. The compound of any one of claims 60, 65, 67, 71 or 72, wherein the N-attached heteroaryl is selected from the group consisting of oxazolyl, thiazolyl, tetrazolyl, imidazolyl, pyrrolyl, pyridyl, pyrimidyl, quinolinyl, and isoquinolinyl, any one of which is optionally substituted.

77-78. (canceled)

79. The compound of claim 71 having the formula 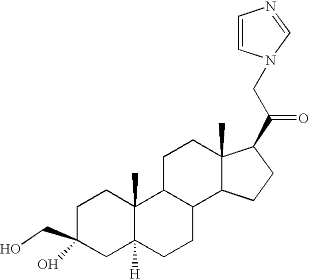
or a pharmaceutically acceptable salt or solvate thereof.

80. The compound of claim 72 having the formula 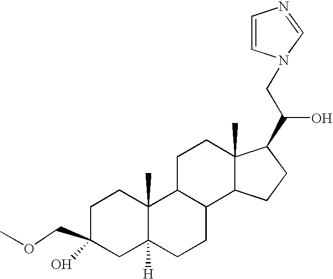
or a pharmaceutically acceptable salt or solvate thereof.

81. A pharmaceutical composition comprising an effective amount of a compound of any one of claims 60, 65, 67, 71, or 72 and one or more pharmaceutically acceptable carrier or diluent.

82. A method for modulating brain excitability by administering an effective amount of a compound of any one of claims 60, 65, 67, 71, or 72 to a mammal in need of such treatment.
